(19) United States
(12) Patent Application Publication
(10) Pub. No.: US 2009/0118288 A1
(43) Pub. Date: May 7, 2009

(54) N-Benzyl-Morpholine Derivatives as Modulators of the Chemokine Receptor

(75) Inventors: Christopher Luckhurst, Leicestershire (GB); Brian Springthorpe, Leicestershire (GB)

Correspondence Address: FISH & RICHARDSON P.C., P.O BOX 1022, MINNEAPOLIS, MN, 55440-1022, US

(73) Assignee: ASTRAZENECA AB, Sodertalje (SE)

(21) Appl. No.: 11/996,131

(22) PCT Filed: Jul. 19, 2006
(86) PCT No.: PCT/SE2006/000892

§ 371 Date: Jun. 17, 2008

(30) Foreign Application Priority Data
Jul. 21, 2005 (SE) 0501718-1

(51) Int. Cl. A61K 31/5377; C07D 413/12; A61K 31/5375; C07D 265/30

(52) U.S. Cl. 514/237.2; 544/124; 544/168; 514/237.8

(57) ABSTRACT

The present invention provides a compound of a formula (I): wherein the variables are defined herein; to a process for preparing such a compound; and to the use of such a compound in the treatment of a chemokine (such as CCR3) mediated disease state.

The present invention concerns N-benzyl-morpholine derivatives having pharmaceutical activity, to processes for preparing such derivatives, to pharmaceutical compositions comprising such derivatives and to the use of such derivatives as active therapeutic agents.

Pharmaceutically active N-benzyl-morpholine derivatives are disclosed in WO 02/26722 or WO 03/082291.

Chemokines are chemotactic cytokines that are released by a wide variety of cells to attract macrophages, T cells, eosinophils, basophils and neutrophils to sites of inflammation and also play a rôle in the maturation of cells of the immune system. Chemokines play an important rôle in immune and inflammatory responses in various diseases and disorders, including asthma and allergic diseases, as well as autoimmune pathologies such as rheumatoid arthritis and atherosclerosis. These small secreted is molecules are a growing superfamily of 8-14 kDa proteins characterised by a conserved four cysteine motif. The chemokine superfamily can be divided into two main groups exhibiting characteristic structural motifs, the Cys-X-Cys (C—X—C, or α) and Cys-Cys (C—C, or β) families. These are distinguished on the basis of a single amino acid insertion between the NH-proximal pair of cysteine residues and sequence similarity.

The C—X—C chemokines include several potent chemoattractants and activators of neutrophils such as interleukin-8 (IL-8) and neutrophil-activating peptide 2 (NAP-2).

The C—C chemokines include potent chemoattractants of monocytes and lymphocytes but not neutrophils such as human monocyte chemotactic proteins 1-3 (MCP-1, MCP-2 and MCP-3), RANTES (Regulated on Activation, Normal T Expressed and Secreted), eotaxin and the macrophage inflammatory proteins 1α and 1β (MIP-1α and MIP-1β).

Studies have demonstrated that the actions of the chemokines are mediated by subfamilies of G protein-coupled receptors, among which are the receptors designated CCR1, CCR2, CCR2A, CCR2B, CCR3, CCR4, CCR5, CCR6, CCR7, CCR8, CCR9, CCR10, CXCR1, CXCR2, CXCR3 and CXCR4. These receptors represent good targets for drug development since agents which modulate these receptors would be useful in the treatment of disorders and diseases such as those mentioned above.

Viral infections are known to cause lung inflammation. It has been shown experimentally that the common cold increases mucosal output of eotaxin in the airways. Instillation of eotaxin into the nose can mimic some of the signs and symptoms of a common cold. (See, Greiff L et al Allergy (1999) 54(11) 1204-8 [Experimental common cold increase mucosal output of eotaxin in atopic individuals] and Kawaguchi M et al Int. Arch. Allergy Immunol. (2000) 122 S1 44 [Expression of eotaxin by normal airway epithelial cells after virus A infection].)

The present invention provides a compound of formula (I):

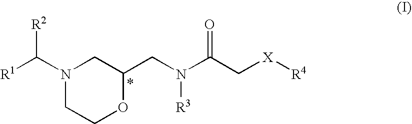

wherein:
R¹ is phenyl optionally substituted by halogen, cyano, C₁₋₄ alkyl or C₁₋₄ alkoxy;
R² and R³ are, independently, hydrogen or C₁₋₆ alkyl;

### X is O, S, S(O) or S(O)₂;

R⁴ is phenyl, naphthyl or heteroaryl substituted with CO₂R and optionally further substituted with halogen, hydroxy, nitro, S(O)_{q}(C₁₋₆ alkyl), S(O)₂NH₂, S(O)₂NH(C₁₋₆ alkyl), S(O)₂N(C₁₋₆ alkyl)₂, NH₂, NH(C₁₋₆ alkyl), N(C₁₋₆ alkyl)₂, cyano, C₁₋₆ alkyl, C₁₋₆ alkoxy, C(O)NH₂, C(O)NH(C₁₋₆ alkyl), C(O)N(C₁₋₆ alkyl)₂, CO₂H, CO₂(C₁₋₆ alkyl), NHC(O)(C₁₋₆ alkyl), NHS(O)₂(C₁₋₆ alkyl), C(O)(C₁₋₆ alkyl), CF₃ or OCF₃;
R is hydrogen, C₁₋₆ alkyl or phenyl(C₁₋₄ alkyl); wherein the phenyl is optionally substituted with halogen, hydroxy, nitro, S(O)ₙ(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃; or CO₂R is (CO₂⁻)ₚR^{P+} wherein R^{P+} is a univalent cation (for example an alkali metal cation) or two carboxylates may coordinate to a divalent cation (for example an alkaline earth metal cation);
n and q are, independently, 0, 1 or 2;
p is 1 or 2;
or a N-oxide thereof; or a pharmaceutically acceptable salt thereof.

Certain compounds of the present invention can exist in different isomeric forms (such as enantiomers, diastereomers, geometric isomers or tautomers). The present invention covers all such isomers and mixtures thereof in all proportions.

The compounds of the invention can be zwitterionic and all such zwitterions are within the invention.

Suitable salts include acid addition salts such as hydrochloride, dihydrochloride, hydrobromide, phosphate, sulfate, acetate, diacetate, fumarate, maleate, malonate, succinate, tartrate, citrate, oxalate, methanesulfonate, benzenesulfonate or p-toluenesulfonate.

An alkali metal cation is, for example sodium or potassium, and an alkaline earth metal cation is, for example, magnesium or calcium.

The compounds of the invention may exist as solvates (such as hydrates) and the present invention covers all such solvates.

Halogen includes fluorine, chlorine, bromine and iodine. Halogen is, for example, fluorine or chlorine.

Alkyl is straight or branched chain and is, for example, methyl, ethyl, n-propyl, isopropyl or tert-butyl.

Heteroaryl is, for example, an aromatic 5 or 6 membered ring, optionally fused to one or more other rings, comprising at least one heteroatom selected from the group comprising nitrogen, oxygen and sulfur; or an N-oxide thereof, or an S-oxide or S-dioxide thereof. Heteroaryl is, for example, furyl, thienyl (also known as thiophenyl), pyrrolyl, thiazolyl, isothiazolyl, pyrazolyl, oxazolyl, isoxazolyl, imidazolyl, [1,2,4]-triazolyl, [1,2,3]-triazolyl, pyridinyl, pyrimidinyl, pyrazinyl, pyridazinyl, indolyl, benzo[b]furyl (also known as benzofuryl), benz[b]thienyl (also known as benzothienyl or benzothiophenyl), indazolyl, benzimidazolyl, benztriazolyl, benzoxazolyl, benzthiazolyl, 1,2,3-benzothiadiazolyl, an imidazopyridinyl (such as imidazo[1,2a]pyridinyl), thieno[3,2-b]pyridin-6-yl, 1,2,3-benzoxadiazolyl (also known as benzo[1,2,3]oxadiazolyl), 1,2,3-benzthiadiazolyl (also known as benzo[1,2,3]thiadiazolyl), 2,1,3-benzothiadiazolyl, benzofurazan (also known as 2,1,3-benzoxadiazolyl), quinoxalinyl, a pyrazolopyridine (for example 1H-pyrazolo[3,4-b]pyridinyl), quinolinyl, isoquinolinyl or a naphthyridinyl (for example [1,6]naphthyridinyl or [1,8]naphthyridinyl); or an N-oxide thereof, or an S-oxide or S-dioxide thereof.

In one particular aspect the present invention provides a compound wherein R¹ is phenyl optionally substituted (for example with one, two or three of the same or different) with fluorine, chlorine, cyano, C₁₋₄ alkyl (for example methyl) or C₁₋₄ alkoxy (for example methoxy).

In another aspect the present invention provides a compound wherein R¹ is phenyl optionally substituted (for example with one, two or three of the same or different) with fluorine, chlorine, cyano or C₁₋₄ alkyl (for example methyl).

In yet another aspect the present invention provides a compound wherein R¹ is phenyl substituted by one, two or three substituents independently selected from: fluorine, chlorine, cyano and methyl.

In a further aspect the present invention provides a compound wherein R¹ is phenyl substituted by one, two or three substituents independently selected from: chlorine and methyl. For example R¹ is 3-chlorophenyl, 4-chlorophenyl, 4-fluorophenyl, 3,4-difluorophenyl, 2-methyl-4-chlorophenyl, 2-methylphenyl, 3,4-dichlorophenyl, 2,4-dichloro-3-methylphenyl or 3,4-dichloro-2-methylphenyl.

In a still further aspect the present invention provides a compound wherein R² is hydrogen.

In another aspect the present invention provides a compound wherein R³ is hydrogen.

In yet another aspect the present invention provides a compound wherein X is S.

In a further aspect the present invention provides a compound wherein X is O.

In a still further aspect the present invention provides a compound wherein R⁴ is phenyl or heteroaryl substituted as described above. Heteroaryl is, for example, furyl, thienyl (also known as thiophenyl), pyrrolyl, thiazolyl, isothiazolyl, pyrazolyl, oxazolyl, isoxazolyl, imidazolyl, [1,2,4]-triazolyl, [1,2,3]-triazolyl, pyridinyl, pyrimidinyl, pyrazinyl, pyridazinyl, indolyl, benzo[b]furyl or benz[b]thienyl.

In another aspect the present invention provides a compound wherein R⁴ is phenyl or pyridinyl substituted as described above.

In yet another aspect the present invention provides a compound wherein R⁴ is phenyl or pyridinyl substituted with CO₂R and optionally further substituted with halogen, S(O)_{q}(C₁₋₆ alkyl), S(O)₂NH₂, NH₂, NH(C₁₋₆ alkyl), N(C₁₋₆ alkyl)₂, C₁₋₆ alkyl, C₁₋₆ alkoxy, C(O)NH₂, C(O)(C₁₋₆ alkyl), CF₃ or OCF₃. Wherein q is 0, 1 or 2.

In a further aspect the present invention provides a compound wherein R⁴ is phenyl or pyridinyl substituted with CO₂R and optionally further substituted with halogen, C₁₋₆ alkyl or CF₃.

In a still further aspect the present invention provides a compound wherein R is hydrogen.

In another aspect the present invention provides a compound wherein R is C₁₋₄ alkyl (for example methyl or ethyl).

In yet another aspect the present invention provides a compound of formula (I), where R³ is hydrogen, having the S stereochemistry at the carbon marked*.

In a further aspect the present invention provides a compound of formula (I) wherein: R¹ is phenyl optionally substituted by halogen (for example chloro); R² and R³ are both hydrogen; X is O or S; R⁴ is phenyl or heteroaryl (for example pyridyl) substituted with CO₂R and optionally further substituted with halogen (such as chloro), C₁₋₆ alkyl (such as tert-butyl) or CF₃; and, R is hydrogen or C₁₋₆ alkyl (for example methyl).

The compounds of the present invention can be prepared as described below or by adaptation of methods described in the art (for example WO 02/26722 or WO 03/082291).

A compound of formula (I) where X is S(O) or S(O)₂ may be prepared by oxidation of a compound of formula (I) where X is S with a suitable oxidant, for example mCPBA in a suitable solvent, for example dichloromethane, at a suitable temperature (such as 0-30° C.).

A compound of formula (I) can be prepared by reacting a compound of formula (II):

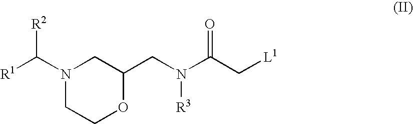

wherein L¹ is a leaving group (for example halogen, such as chloro), with a compound R⁴XH in the presence of a suitable buffer (for example sodium acetate) in a suitable solvent (such as an aliphatic alcohol, for example ethanol) at a suitable temperature (such as 50-120° C., for example reflux).

Alternatively, a compound of formula (I) can be prepared by reacting a compound of formula (III):

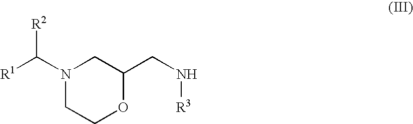

with a compound of formula (IV):

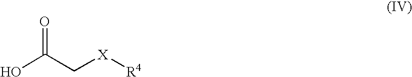

in the presence of a suitable coupling agent (such as HATU), in the presence of a suitable base (such as a tertiary amine, for example Hünig's base), in a suitable solvent (such as N-methylpyrrolidinone) at a temperature in the range −10 to 30° C.

Alternatively, a compound of formula (I), wherein CO₂R is an ester, can be prepared by reacting a compound of formula (V):

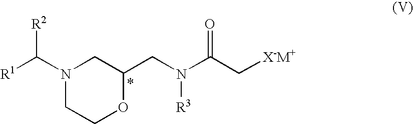

wherein M⁺ is an alkali metal cation (such as sodium, lithium or potassium), with L²-R⁴, wherein L² is a leaving group (for example halogen, such as fluoro), in a suitable solvent (such as DMF) at ambient temperature (0-30° C.).

For a compound of formula (I):
wherein R is hydrogen said compound may be converted to a compound of the invention where CO₂R is an ester by a standard esterification method well known in the art;
wherein CO₂R is an ester said compound may be converted to a compound of the invention where R is hydrogen by a standard ester hydrolysis method well known in the art; and,
wherein CO₂R is CO₂⁻R⁺ said compound can be prepared by reacting a compound wherein R is hydrogen or alkyl or substituted alkyl, with a suitable alkali metal or alkaline earth metal hydroxide.
   Such methods are described in undergraduate organic chemistry textbooks (such as Advanced Organic Chemistry by J March, 5^{th} edition M B Smith and J March, Wiley, 2001).

Compounds of formula (III) wherein R² and R³ are both hydrogen, and the carbon marked * has the S absolute stereochemistry are important intermediates for the preparation of compounds of formula (I) and also for compounds disclosed in WO 02/26722 or WO 03/082291. As yet no stereospecific synthesis for such compounds has been disclosed. The method for obtaining active ingredients disclosed in WO 02/26722 and WO 03/082291 is the separation of a racemic mixture into its component single enantiomers using preparative chiral-HPLC. This is not an optimal way of obtaining such compounds as the unwanted R-isomer has to be disposed of, and, therefore, starting materials are not used efficiently and effectively.

Thus in a further aspect the present invention provides a process for the preparation of a compound of formula (IIIA):

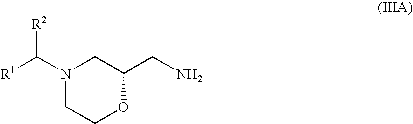

the process comprising reduction of a compound of formula (VI):

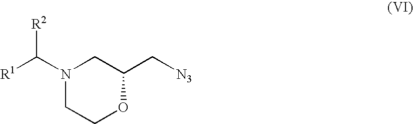

for example with triphenyl phosphine in the presence of water in a suitable solvent (for example tetrahydrofuran) at a suitable temperature (for example 60-100° C., such as reflux). Alternative reduction methods for azides, such as hydrogenation or reaction with a disulfide are well-known to those skilled in the art.

A compound of formula (VI) can be prepared by reacting a compound of formula (VII):

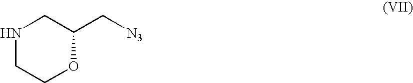

with an aldehyde of formula R¹CHO or a ketone of formula R¹COR² in the presence of a suitable amine (such as a tri(C₁₋₆ alkyl)amine for example triethylamine or Hünig's base), a suitable reducing agent (such as sodium triacetoxyborohydride or sodium cyanoborohydride) in the presence of a suitable acid (such as a C₁₋₆ aliphatic acid, for example acetic acid) at a suitable temperature (for example 0-30° C.).

A compound of formula (VII) can be prepared by deprotecting a compound of formula (VIIa):

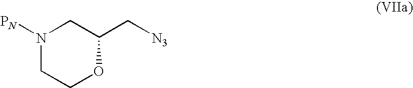

using a method known in the art.

A compound of formula (VIIa) can be prepared by reacting a compound of formula (VIII):

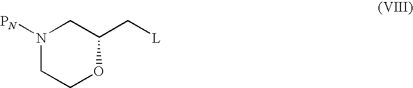

wherein L is a suitable leaving group {for example an aryl sulfonate (such as benzene sulfonate or toluenesulfonate), a C₁₋₆ alkylsulfonate (such as methylsulfonate), triflate or a halide (such as chloride or bromide)} with an alkali metal azide (such as sodium azide), optionally in the presence of an iodide (such as lithium iodide) in a suitable solvent (such as DMF) at a suitable temperature (such as 40-80° C.).

Alternatively a different synthetic equivalent of ammonia can be used, for example compound (VIII) can be reacted with potassium phthalimide or with ammonia, followed by a suitable protection step. In these cases the group P_{N} needs to be removed, and the group R¹CH₂ added under conditions that do not affect the protecting group on the other nitrogen atom. Subsequent deprotection will reveal a compound of formula (IIIA).

A compound of formula (VIII) can be prepared by reacting a compound of formula (IX):

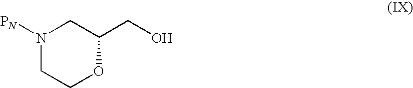

in a method known in the art. For example to prepare a compound wherein L is methanesulfonate then react a compound of formula (IX) with methanesulfonyl chloride in the presence of a suitable base (such as a tri(C₁₋₆ alkyl)amine for example triethylamine or Hünig's base), in a suitable solvent (such as DCM) at a suitable temperature (for example in the range −10 to 20° C.).

A compound of formula (IX) can be prepared by deprotecting a compound of formula (X):

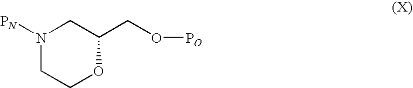

Wherein P_{O} is as defined below using a method known in the art.

A compound of formula (X) can be prepared by protecting a compound of formula (XI):

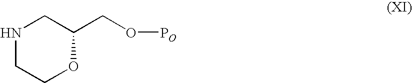

with P_{N}L, wherein L is a leaving group, under conditions known in the art.

A compound of formula (XI) can be prepared by reacting a compound of formula (XII):

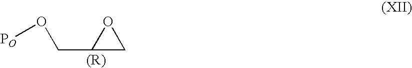

with 2-aminomethane or a salt thereof (such as the hydrogen sulphate salt) in water and, when a salt is used, in the presence of a suitable base (such as sodium hydroxide), at a temperature in the range 20-80° C.

A person skilled in the art will appreciate that the protecting groups P_{O} and P_{N} must be chosen so that one can be removed in the presence of the other (that is they must be orthogonal). For example the protecting groups P_{O} and P_{N} can be chosen according to the following table:

| P_{O} | P_{N} |
|---|---|
| Cleavable by Hydrogenolysis: | Cleavable by acid: |
| For example benzyl, substituted benzyl, | For example BOC; methyl carbamate, |
| benzhydryl, carbobenzyloxy | amides (like benzamide, acetamide), |
| | sulfonamides (like benzenesulfonamide) |
| | Cleavable by amines |
| | For example FMOC |
| | Cleavable by base: |
| | For example trifluoroaceamide |
| | Cleavable by zinc-acetic reduction: |
| | For example trichloroethylcarbamate |
| Cleavable by basic hydrolysis: | Cleavable by acid: |
| For example acetate, pivaloate, benzoate, methyl | For example BOC; methyl carbamate, |
| carbonate, FMOC | amides (like benzamide, acetamide), |
| | sulfonamides (like benzenesulfonamide) |
| | Cleavable by zinc-acetic reduction: |
| | For example trichloroethylcarbamate |
| | Cleavable by hydrogenolysis: |
| | For example carbobenzyloxy |
| Cleavable by zinc-acetic reduction: | Cleavable by acid: |
| For example trichloroethylcarbonate | For example BOC; methyl carbamate, |
| | amides (like benzamide, acetamide), |
| | sulfonamides (like benzenesulfonamide) |
| | Cleavable by amines |
| | For example FMOC |
| | Cleavable by base: |
| | For example trifluoroaceamide |
| | Cleavable by hydrogenolysis: |
| | For example carbobenzyloxy |
| Cleavable by acid: | Cleavable by amines |
| For example BOC; methyl carbonate, esters | For example FMOC |
| (like benzoate, acetate, pivaloate) | |
| | Cleavable by base: |
| | For example trifluoroaceamide |
| | Cleavable by zinc-acetic reduction: |
| | For example trichloroethylcarbamate |
| | Cleavable by hydrogenolysis: |
| | For example carbobenzyloxy |
| No protecting group | Cleavable by acid: |
| | For example BOC; methyl carbamate, |
| | amides (like benzamide, acetamide), |
| | sulfonamides (like benzenesulfonamide) |
| | Cleavable by amines |
| | For example FMOC |
| | Cleavable by base: |
| | For example trifluoroaceamide |
| | Cleavable by zinc-acetic reduction: |
| | For example trichloroethylcarbamate |
| | Cleavable by hydrogenolysis: |
| | For example carbobenzyloxy |
| Cleavable by fluoride | Cleavable by amines |
| For example TIPS, TBDPS | For example FMOC |
| | Cleavable by base: |
| | For example trifluoroaceamide |
| | Cleavable by zinc-acetic reduction: |
| | For example trichloroethylcarbamate |
| | Cleavable by hydrogenolysis: |
| | For example carbobenzyloxy |

Alternatively a compound of formula (III) wherein R² is H may be prepared by reduction of a compound of formula (XIII)

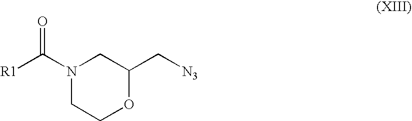

with a suitable reducing agent (for example borane) in a suitable solvent, for example tetrahydrofuran at a suitable temperature, for example reflux.

A compound of formula (XIII) may be prepared by acylation of a compound of formula (V) with an acid of formula (XIV) in the presence of a suitable coupling agent or an acid chloride of formula (XV) in the presence of a base.

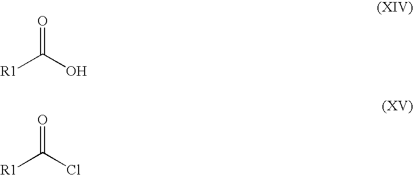

The preparations of various intermediates are described in the literature or can be prepared by routine adaptation of methods described in the literature.

In the above processes it may be desirable or necessary to protect an acid group or a hydroxy or other potentially reactive group. Suitable protecting groups and details of processes for adding and removing such groups may be found in “Protective Groups in Organic Synthesis”, 3rd Edition (1999) by Greene and Wuts.

In another aspect the present invention provides processes for the preparation of compounds of formula (I).

The compounds of formula (I) have activity as pharmaceuticals, in particular as modulators of chemokine receptor (for example CCR3) activity, and may be used in the treatment of autoimmune, inflammatory, proliferative or hyperproliferative diseases, or immunologically-mediated diseases (including rejection of transplanted organs or tissues and Acquired Immunodeficiency Syndrome (AIDS)).

Examples of these conditions are:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) or adenovirus; or eosinophilic esophagitis;
2. bone and joints: arthritides associated with or including osteoarthritis/osteoarthrosis, both primary and secondary to, for example, congenital hip dysplasia; cervical and lumbar spondylitis, and low back and neck pain; osteoporosis; rheumatoid arthritis and Still's disease; seronegative spondyloarthropathies including ankylosing spondylitis, psoriatic arthritis, reactive arthritis and undifferentiated spondarthropathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositis and polymyositis; polymyalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthralgias, tendonititides, and myopathies;
3. pain and connective tissue remodelling of musculoskeletal disorders due to injury [for example sports injury] or disease: arthitides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritis, scleroderma, mixed connective tissue disorder, spondyloarthropathies or periodontal disease (such as periodontitis);
4. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen scleroses et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia greata, male-pattern baldness, Sweet's syndrome, Weber-Christian syndrome, erythema multiform; cellulitis, both infective and non-infective; panniculitis; cutaneous lymphomas, non-melanoma skin cancer and other dysplastic lesions; drug-induced disorders including fixed drug eruptions;
5. eyes: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial;
6. gastrointestinal tract: glossitis, gingivitis, periodontitis; esophagitis, including reflux; eosinophilic gastro-enteritis, mastocytosis, Crohn's disease, colitis including ulcerative colitis, proctitis, pruritus ani; coeliac disease, irritable bowel syndrome, and food-related allergies which may have effects remote from the gut (for example migraine, rhinitis or eczema);
7. abdominal: hepatitis, including autoimmune, alcoholic and viral; fibrosis and cirrhosis of the liver; cholecystitis; pancreatitis, both acute and chronic;
8. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);
9. allograft rejection: acute and chronic following, for example, transplantation of kidney, heart, liver, lung, bone marrow, skin or cornea or following blood transfusion; or chronic graft versus host disease;
10. CNS: Alzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes;
11. other auto-immune and allergic disorders including Hashimoto's thyroiditis, Graves' disease, Addison's disease, diabetes mellitus, idiopathic thrombocytopenic purpura, eosinophilic fascitis, hyper-IgE syndrome, antiphospholipid syndrome;
12. other disorders with an inflammatory or immunological component; including acquired immune deficiency syndrome (AIDS), leprosy, Sezary syndrome, and paraneoplastic syndromes;
13. cardiovascular: atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (for example syphilitic); vasculitides; disorders of the proximal and peripheral veins including phlebitis and thrombosis, including deep vein thrombosis and complications of varicose veins;
14. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; or,
15. gastrointestinal tract: Coeliac disease, proctitis, eosinophilic gastro-enteritis, mastocytosis, Crohn's disease, ulcerative colitis, microscopic colitis, indeterminant colitis, irritable bowel disorder, irritable bowel syndrome, non-inflammatory diarrhea, food-related allergies which have effects remote from the gut, e.g., migraine, rhinitis and eczema.

According to a further feature of the present invention there is provided a method for treating a chemokine mediated disease state (for example a CCR3 mediated disease state) in a mammal, such as man, suffering from, or at risk of, said disease state, which comprises administering to a mammal in need of such treatment a therapeutically effective amount of a compound of the formula (I) or a pharmaceutically acceptable salt thereof.

According to yet another feature of the present invention there is provided a method for treating a sign and/or symptom of what is commonly referred to as a cold in a mammal, such as man, suffering from, or at risk of, said disease state, which comprises administering to a mammal in need of such treatment a therapeutically effective amount of a compound of the formula (I) or a pharmaceutically acceptable salt thereof.

The invention also provides a compound of the formula (I), or a pharmaceutically acceptable salt thereof, for use in therapy.

In another aspect the invention provides the use of a compound of formula (I), or a pharmaceutically acceptable salt thereof, in the manufacture of a medicament for use in therapy (for example modulating chemokine receptor activity (for example CCR3 receptor activity) or treating a sign and/or symptom of what is commonly referred to as a cold).

The invention further provides the use of a compound of formula (I), or a pharmaceutically acceptable salt thereof, in the manufacture of a medicament for use in the treatment of:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) or adenovirus; or eosinophilic esophagitis;
2. bone and joints: arthritides associated with or including osteoarthritis/osteoarthrosis, both primary and secondary to, for example, congenital hip dysplasia; cervical and lumbar spondylitis, and low back and neck pain; osteoporosis; rheumatoid arthritis and Still's disease; seronegative spondyloarthropathies including ankylosing spondylitis, psoriatic arthritis, reactive arthritis and undifferentiated spondarthropathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositis and polymyositis; polymyalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthralgias, tendonititides, and myopathies;
3. pain and connective tissue remodelling of musculoskeletal disorders due to injury [for example sports injury] or disease: arthitides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritis, scleroderma, mixed connective tissue disorder, spondyloarthropathies or periodontal disease (such as periodontitis);
4. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen scleroses et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia greata, male-pattern baldness, Sweet's syndrome, Weber-Christian syndrome, erythema multiform; cellulitis, both infective and non-infective; panniculitis; cutaneous lymphomas, non-melanoma skin cancer and other dysplastic lesions; drug-induced disorders including fixed drug eruptions;
5. eyes: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial;
6. gastrointestinal tract: glossitis, gingivitis, periodontitis; esophagitis, including reflux; eosinophilic gastro-enteritis, mastocytosis, Crohn's disease, colitis including ulcerative colitis, proctitis, pruritus ani; coeliac disease, irritable bowel syndrome, and food-related allergies which may have effects remote from the gut (for example migraine, rhinitis or eczema);
7. abdominal: hepatitis, including autoimmune, alcoholic and viral; fibrosis and cirrhosis of the liver; cholecystitis; pancreatitis, both acute and chronic;
8. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);
9. allograft rejection: acute and chronic following, for example, transplantation of kidney, heart, liver, lung, bone marrow, skin or cornea or following blood transfusion; or chronic graft versus host disease;
10. CNS: Alzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes;
11. other auto-immune and allergic disorders including Hashimoto's thyroiditis, Graves' disease, Addison's disease, diabetes mellitus, idiopathic thrombocytopenic purpura, eosinophilic fascitis, hyper-IgE syndrome, antiphospholipid syndrome;
12. other disorders with an inflammatory or immunological component; including acquired immune deficiency syndrome (AIDS), leprosy, Sezary syndrome, and paraneoplastic syndromes;
13. cardiovascular: atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (for example syphilitic); vasculitides; disorders of the proximal and peripheral veins including phlebitis and thrombosis, including deep vein thrombosis and complications of varicose veins;
14. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; or,
15. gastrointestinal tract: Coeliac disease, proctitis, eosinophilic gastro-enteritis, mastocytosis, Crohn's disease, ulcerative colitis, microscopic colitis, indeterminant colitis, irritable bowel disorder, irritable bowel syndrome, non-inflammatory diarrhea, food-related allergies which have effects remote from the gut, e.g., migraine, rhinitis and eczema;
in a mammal (for example man).

In a further aspect the invention provides a compound of formula (I), or a pharmaceutically acceptable salt thereof, for use in the treatment of asthma {such as bronchial, allergic, intrinsic, extrinsic or dust asthma, particularly chronic or inveterate asthma (for example late asthma or airways hyper-responsiveness)}; or rhinitis {including acute, allergic, atrophic or chronic rhinitis, such as rhinitis caseosa, hypertrophic rhinitis, rhinitis purulenta, rhinitis sicca or rhinitis medicamentosa; membranous rhinitis including croupous, fibrinous or pseudomembranous rhinitis or scrofulous rhinitis; seasonal rhinitis including rhinitis nervosa (hay fever) or vasomotor rhinitis}.

In a still further aspect a compound of formula (I), or a pharmaceutically acceptable salt thereof, is useful in the treatment of asthma.

In another aspect a compound of formula (I), or a pharmaceutically acceptable salt thereof, is useful in the treatment of respiratory syncytial virus.

The present invention also provides a the use of a compound of formula (I), or a pharmaceutically acceptable salt thereof, in the manufacture of a medicament for use in the treatment of asthma {such as bronchial, allergic, intrinsic, extrinsic or dust asthma, particularly chronic or inveterate asthma (for example late asthma or airways hyper-responsiveness)}; or rhinitis {including acute, allergic, atrophic or chronic rhinitis, such as rhinitis caseosa, hypertrophic rhinitis, rhinitis purulenta, rhinitis sicca or rhinitis medicamentosa; membranous rhinitis including croupous, fibrinous or pseudomembranous rhinitis or scrofulous rhinitis; seasonal rhinitis including rhinitis nervosa (hay fever) or vasomotor rhinitis}.

In order to use a compound of the invention, or a pharmaceutically acceptable salt thereof, for the therapeutic treatment of a mammal, such as man, said ingredient is normally formulated in accordance with standard pharmaceutical practice as a pharmaceutical composition. Therefore in another aspect the present invention provides a pharmaceutical composition which comprises a compound of the formula (I), or a pharmaceutically acceptable salt thereof (active ingredient), and a pharmaceutically acceptable adjuvant, diluent or carrier.

In a further aspect the present invention provides a process for the preparation of said composition which comprises mixing active ingredient with a pharmaceutically acceptable adjuvant, diluent or carrier. Depending on the mode of administration, the pharmaceutical composition will, for example, comprise from 0.05 to 99% w (percent by weight), such as from 0.05 to 80% w, for example from 0.10 to 70% w, such as from 0.10 to 50% w, of active ingredient, all percentages by weight being based on total composition.

The pharmaceutical compositions of this invention may be administered in standard manner for the disease condition that it is desired to treat, for example by topical (such as to the lung and/or airways or to the skin), oral, rectal or parenteral administration. For these purposes the compounds of this invention may be formulated by means known in the art. A suitable pharmaceutical composition of this invention is one suitable for oral administration in unit dosage form, for example a tablet or capsule which contains between 0.1 mg and 1 g of active ingredient.

Each patient may receive, for example, a dose of 0.01 mgkg⁻¹ to 100 mgkg⁻¹, for example in the range of 0.1 mgkg⁻¹ to 20 mgkg⁻¹, of the active ingredient administered, for example, 1 to 4 times per day.

The invention further relates to a combination therapy wherein a compound of the invention, or a pharmaceutically acceptable salt thereof, or a pharmaceutical composition or formulation comprising a compound of the invention, is administered concurrently or sequentially or as a combined preparation with another therapeutic agent or agents, for the treatment of one or more of the conditions listed.

In particular, for the treatment of the inflammatory diseases such as (but not restricted to) rheumatoid arthritis, osteoarthritis, asthma, allergic rhinitis, chronic obstructive pulmonary disease (COPD), psoriasis, and inflammatory bowel disease, the compounds of the invention may be combined with agents listed below.

Non-steroidal anti-inflammatory agents (hereinafter NSAIDs) including non-selective cyclo-oxygenase COX-1/COX-2 inhibitors whether applied topically or systemically (such as piroxicam, diclofenac, propionic acids such as naproxen, flurbiprofen, fenoprofen, ketoprofen and ibuprofen, fenamates such as mefenamic acid, indomethacin, sulindac, azapropazone, pyrazolones such as phenylbutazone, salicylates such as aspirin); selective COX-2 inhibitors (such as meloxicam, celecoxib, rofecoxib, valdecoxib, lumarocoxib, parecoxib and etoricoxib); cyclo-oxygenase inhibiting nitric oxide donors (CINODs); glucocorticosteroids (whether administered by topical, oral, intramuscular, intravenous, or intra-articular routes); methotrexate; leflunomide; hydroxychloroquine; d-penicillamine; auranofin or other parenteral or oral gold preparations; analgesics; diacerin; intra-articular therapies such as hyaluronic acid derivatives; and nutritional supplements such as glucosamine.

The present invention still further relates to the combination of a compound of the invention, or a pharmaceutically acceptable salt thereof, together with a cytokine or agonist or antagonist of cytokine function, (including agents which act on cytokine signalling pathways such as modulators of the SOCS system) including alpha-, beta-, and gamma-interferons; insulin-like growth factor type I (IGF-1); interleukins (IL) including IL1 to 17, and interleukin antagonists or inhibitors such as anakinra; tumour necrosis factor alpha (TNF-α) inhibitors such as anti-TNF monoclonal antibodies (for example infliximab; adalimumab, and CDP-870) and TNF receptor antagonists including immunoglobulin molecules (such as etanercept) and low-molecular-weight agents such as pentoxifylline.

In addition the invention relates to a combination of a compound of the invention, or a pharmaceutically acceptable salt thereof, with a monoclonal antibody targeting B-Lymphocytes (such as CD20 (rituximab), MRA-aIL16R and T-Lymphocytes, CTLA4-Ig, HuMax Il-15).

The present invention still further relates to the combination of a compound of the invention, or a pharmaceutically acceptable salt thereof, with a modulator of chemokine receptor function such as an antagonist of CCR1, CCR2, CCR2A, CCR2B, CCR3, CCR4, CCR5, CCR6, CCR7, CCR8, CCR9, CCR10 and CCR11 (for the C—C family); CXCR1, CXCR2, CXCR3, CXCR4 and CXCR5 (for the C—X—C family) and CX₃CR1 for the C—X₃—C family.

The present invention further relates to the combination of a compound of the invention, or a pharmaceutically acceptable salt thereof, with an inhibitor of matrix metalloprotease (MMPs), i.e., the stromelysins, the collagenases, and the gelatinases, as well as aggrecanase; for example collagenase-1 (MMP-1), collagenase-2 (MMP-8), collagenase-3 (MMP-13), stromelysin-1 (MMP-3), stromelysin-2 (MMP-10), and stromelysin-3 (MMP-11) and MMP-9 and MMP-12, including agents such as doxycycline.

The present invention still further relates to the combination of a compound of the invention, or a pharmaceutically acceptable salt thereof, and a leukotriene biosynthesis inhibitor, 5-lipoxygenase (5-LO) inhibitor or 5-lipoxygenase activating protein (FLAP) antagonist such as; zileuton; ABT-761; fenleuton; tepoxalin; Abbott-79175; Abbott-85761; a N-(5-substituted)-thiophene-2-alkylsulfonamide; 2,6-di-tert-butylphenolhydrazones; a methoxytetrahydropyrans such as Zeneca ZD-2138; the compound SB-210661; a pyridinyl-substituted 2-cyanonaphthalene compound such as L-739,010; a 2-cyanoquinoline compound such as L-746,530; or an indole or quinoline compound such as MK-591, MK-886, and BAY×1005.

The present invention further relates to the combination of a compound of the invention, or a pharmaceutically acceptable salt thereof, and a receptor antagonist for leukotrienes (LT) B4, LTC4, LTD4, and LTE4. selected from the group consisting of the phenothiazin-3-yls such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY×7195.

The present invention still further relates to the combination of a compound of the invention, or a pharmaceutically acceptable salt thereof, and a phosphodiesterase (PDE) inhibitor such as a methylxanthanine including theophylline and aminophylline; a selective PDE isoenzyme inhibitor including a PDE4 inhibitor an inhibitor of the isoform PDE4D, or an inhibitor of PDE5.

The present invention further relates to the combination of a compound of the invention, or a pharmaceutically acceptable salt thereof, and a histamine type 1 receptor antagonist such as cetirizine, loratadine, desloratadine, fexofenadine, acrivastine, terfenadine, astemizole, azelastine, levocabastine, chlorpheniramine, promethazine, cyclizine, or mizolastine; applied orally, topically or parenterally.

The present invention still further relates to the combination of a compound of the invention, or a pharmaceutically acceptable salt thereof, and a proton pump inhibitor (such as omeprazole) or a gastroprotective histamine type 2 receptor antagonist.

The present invention further relates to the combination of a compound of the invention, or a pharmaceutically acceptable salt thereof, and an antagonist of the histamine type 4 receptor.

The present invention still further relates to the combination of a compound of the invention, or a pharmaceutically acceptable salt thereof, and an alpha-1/alpha-2 adrenoceptor agonist vasoconstrictor sympathomimetic agent, such as propylhexedrine, phenylephrine, phenylpropanolamine, ephedrine, pseudoephedrine, naphazoline hydrochloride, oxymetazoline hydrochloride, tetrahydrozoline hydrochloride, xylometazoline hydrochloride, tramazoline hydrochloride or ethylnorepinephrine hydrochloride.

The present invention further relates to the combination of a compound of the invention, or a pharmaceutically acceptable salt thereof, and an anticholinergic agent including muscarinic receptor (M1, M2, and M3) antagonist such as atropine, hyoscine, glycopyrrrolate, ipratropium bromide, tiotropium bromide, oxitropium bromide, pirenzepine or telenzepine.

The present invention still further relates to the combination of a compound of the invention, or a pharmaceutically acceptable salt thereof, and a beta-adrenoceptor agonist (including beta receptor subtypes 1-4) such as isoprenaline, salbutamol, formoterol, salmeterol, terbutaline, orciprenaline, bitolterol mesylate, or pirbuterol, or a chiral enantiomer thereof.

The present invention further relates to the combination of a compound of the invention, or a pharmaceutically acceptable salt thereof, and a chromone, such as sodium cromoglycate or nedocromil sodium.

The present invention still further relates to the combination of a compound of the invention, or a pharmaceutically acceptable salt thereof, with a glucocorticoid, such as flunisolide, triamcinolone acetonide, beclomethasone dipropionate, budesonide, fluticasone propionate, ciclesonide or mometasone furoate.

The present invention further relates to the combination of a compound of the invention, or a pharmaceutically acceptable salt thereof, with an agent that modulates a nuclear hormone receptor such as PPARs.

The present invention still further relates to the combination of a compound of the invention, or a pharmaceutically acceptable salt thereof, together with an immunoglobulin (Ig) or Ig preparation or an antagonist or antibody modulating Ig function such as anti-IgE (for example omalizumab).

The present invention further relates to the combination of a compound of the invention, or a pharmaceutically acceptable salt thereof, and another systemic or topically-applied anti-inflammatory agent, such as thalidomide or a derivative thereof, a retinoid, dithranol or calcipotriol.

The present invention still further relates to the combination of a compound of the invention, or a pharmaceutically acceptable salt thereof, and combinations of aminosalicylates and sulfapyridine such as sulfasalazine, mesalazine, balsalazide, and olsalazine; and immunomodulatory agents such as the thiopurines, and corticosteroids such as budesonide.

The present invention further relates to the combination of a compound of the invention, or a pharmaceutically acceptable salt thereof, together with an antibacterial agent such as a penicillin derivative, a tetracycline, a macrolide, a beta-lactam, a fluoroquinolone, metronidazole, an inhaled aminoglycoside; an antiviral agent including acyclovir, famciclovir, valaciclovir, ganciclovir, cidofovir, amantadine, rimantadine, ribavirin, zanamavir and oseltamavir; a protease inhibitor such as indinavir, nelfmavir, ritonavir, and saquinavir; a nucleoside reverse transcriptase inhibitor such as didanosine, lamivudine, stavudine, zalcitabine or zidovudine; or a non-nucleoside reverse transcriptase inhibitor such as nevirapine or efavirenz.

The present invention still further relates to the combination of a compound of the invention, or a pharmaceutically acceptable salt thereof, and a cardiovascular agent such as a calcium channel blocker, a beta-adrenoceptor blocker, an angiotensin-converting enzyme (ACE) inhibitor, an angiotensin-2 receptor antagonist; a lipid lowering agent such as a statin or a fibrate; a modulator of blood cell morphology such as pentoxifylline; thrombolytic, or an anticoagulant such as a platelet aggregation inhibitor.

The present invention further relates to the combination of a compound of the invention, or a pharmaceutically acceptable salt thereof, and a CNS agent such as an antidepressant (such as sertraline), an anti-Parkinsonian drug (such as deprenyl, L-dopa, ropinirole, pramipexole, a MAOB inhibitor such as selegine and rasagiline, a comP inhibitor such as tasmar, an A-2 inhibitor, a dopamine reuptake inhibitor, an NMDA antagonist, a nicotine agonist, a dopamine agonist or an inhibitor of neuronal nitric oxide synthase), or an anti-Alzheimer's drug such as donepezil, rivastigmine, tacrine, a COX-2 inhibitor, propentofylline or metrifonate.

The present invention still further relates to the combination of a compound of the invention, or a pharmaceutically acceptable salt thereof, and an agent for the treatment of acute or chronic pain, such as a centrally or peripherally-acting analgesic (for example an opioid or derivative thereof), carbamazepine, phenyloin, sodium valproate, amitryptyline or other anti-depressant agents, paracetamol, or a non-steroidal anti-inflammatory agent.

The present invention further relates to the combination of a compound of the invention, or a pharmaceutically acceptable salt thereof, together with a parenterally or topically-applied (including inhaled) local anaesthetic agent such as lignocaine or a derivative thereof.

A compound of the present invention, or a pharmaceutically acceptable salt thereof, can also be used in combination with an anti-osteoporosis agent including a hormonal agent such as raloxifene, or a biphosphonate such as alendronate.

The present invention still further relates to the combination of a compound of the invention, or a pharmaceutically acceptable salt thereof, together with a: (i) tryptase inhibitor; (ii) platelet activating factor (PAF) antagonist; (iii) interleukin converting enzyme (ICE) inhibitor; (iv) IMPDH inhibitor; (v) adhesion molecule inhibitors including VLA-4 antagonist; (vi) cathepsin; (vii) kinase inhibitor such as an inhibitor of tyrosine kinase (such as Btk, Itk, Jak3 or MAP, for example Gefitinib or Imatinib mesylate), a serine/threonine kinase (such as an inhibitor of a MAP kinase such as p38, JNK, protein kinase A, B or C, or IKK), or a kinase involved in cell cycle regulation (such as a cyclin dependent kinase); (viii) glucose-6 phosphate dehydrogenase inhibitor; (ix) kinin-B.sub1.- or B.sub2.-receptor antagonist; (x) anti-gout agent, for example colchicine; (xi) xanthine oxidase inhibitor, for example allopurinol; (xii) uricosuric agent, for example probenecid, sulfinpyrazone or benzbromarone; (xiii) growth hormone secretagogue; (xiv) transforming growth factor (TGFβ); (xv) platelet-derived growth factor (PDGF); (xvi) fibroblast growth factor for example basic fibroblast growth factor (bFGF); (xvii) granulocyte macrophage colony stimulating factor (GM-CSF); (xviii) capsaicin cream; (xix) tachykinin NK.sub 1. or NK.sub3. receptor antagonist such as NKP-608C, SB-233412 (talnetant) or D-4418; (xx) elastase inhibitor such as UT-77 or ZD-0892; (xxi) TNF-alpha converting enzyme inhibitor (TACE); (xxii) induced nitric oxide synthase (iNOS) inhibitor; (xxiii) chemoattractant receptor-homologous molecule expressed on TH2 cells, (such as a CRTH2 antagonist); (xxiv) inhibitor of p38; (xxv) agent modulating the function of Toll-like receptors (TLR), (xxvi) agent modulating the activity of purinergic receptors such as P2X7; (xxvii) inhibitor of transcription factor activation such as NFkB, API, or STATS; or (xxviii) a non-steroidal glucocorticoid receptor (GR) agonist.

A compound of the invention, or a pharmaceutically acceptable salt thereof, can also be used in combination with an existing therapeutic agent for the treatment of cancer, for example suitable agents include:

(i) an antiproliferative/antineoplastic drug or a combination thereof, as used in medical oncology, such as an alkylating agent (for example cis-platin, carboplatin, cyclophosphamide, nitrogen mustard, melphalan, chlorambucil, busulphan or a nitrosourea); an antimetabolite (for example an antifolate such as a fluoropyrimidine like 5-fluorouracil or tegafur, raltitrexed, methotrexate, cytosine arabinoside, hydroxyurea, gemcitabine or paclitaxel); an antitumour antibiotic (for example an anthracycline such as adriamycin, bleomycin, doxorubicin, daunomycin, epirubicin, idarubicin, mitomycin-C, dactinomycin or mithramycin); an antimitotic agent (for example a vinca alkaloid such as vincristine, vinblastine, vindesine or vinorelbine, or a taxoid such as taxol or taxotere); or a topoisomerase inhibitor (for example an epipodophyllotoxin such as etoposide, teniposide, amsacrine, topotecan or a camptothecin);
(ii) a cytostatic agent such as an antiestrogen (for example tamoxifen, toremifene, raloxifene, droloxifene or iodoxyfene), an oestrogen receptor down regulator (for example fulvestrant), an antiandrogen (for example bicalutamide, flutamide, nilutamide or cyproterone acetate), a LHRH antagonist or LHRH agonist (for example goserelin, leuprorelin or buserelin), a progestogen (for example megestrol acetate), an aromatase inhibitor (for example as anastrozole, letrozole, vorazole or exemestane) or an inhibitor of 5α-reductase such as finasteride;
(iii) an agent which inhibits cancer cell invasion (for example a metalloproteinase inhibitor like marimastat or an inhibitor of urokinase plasminogen activator receptor function);
(iv) an inhibitor of growth factor function, for example: a growth factor antibody (for example the anti-erb b2 antibody trastuzumab, or the anti-erb b1 antibody cetuximab [C225]), a farnesyl transferase inhibitor, a tyrosine kinase inhibitor or a serine/threonine kinase inhibitor, an inhibitor of the epidermal growth factor family (for example an EGFR family tyrosine kinase inhibitor such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) or 6-acrylamido-N-(3-chloro-4-fluorophenyl)-7-(3-morpholinopropoxy)quinazolin-4-amine (CI 1033)), an inhibitor of the platelet-derived growth factor family, or an inhibitor of the hepatocyte growth factor family;
(v) an antiangiogenic agent such as one which inhibits the effects of vascular endothelial growth factor (for example the anti-vascular endothelial cell growth factor antibody bevacizumab, a compound disclosed in WO 97/22596, WO 97/30035, WO 97/32856 or WO 98/13354), or a compound that works by another mechanism (for example linomide, an inhibitor of integrin αvβ3 function or an angiostatin);
(vi) a vascular damaging agent such as combretastatin A4, or a compound disclosed in WO 99/02166, WO 00/40529, WO 00/41669, WO 01/92224, WO 02/04434 or WO 02/08213;
(vii) an agent used in antisense therapy, for example one directed to one of the targets listed above, such as ISIS 2503, an anti-ras antisense;
(viii) an agent used in a gene therapy approach, for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; or,
(ix) an agent used in an immunotherapeutic approach, for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell energy, approaches using transfected immune cells such as cytokine-transfected dendritic cells, approaches using cytokine-transfected tumour cell lines and approaches using anti-idiotypic antibodies.

The invention will now be illustrated by the following non-limiting examples in which, unless stated otherwise:

(i) when given, ¹H NMR data is quoted and is in the form of delta values for major diagnostic protons, given in parts per million (ppm) relative to tetramethylsilane (TMS) as an internal standard, determined at 300 MHz or 400 MHz using perdeuterio DMSO-D6 (CD₃SOCD₃) or CDCl₃ as the solvent unless otherwise stated;
(ii) mass spectra (MS) were run with an electron energy of 70 electron volts in the chemical ionisation (CI) mode using a direct exposure probe; where indicated ionisation was effected by electron impact (EI) or fast atom bombardment (FAB); where values for m/z are given, generally only ions which indicate the parent mass are reported, and unless otherwise stated the mass ion quoted is the positive mass ion-(M+H)⁺;
(iii) the title and sub-title compounds of the examples and preparations were named using the index name program from Ogham and stereochemical descriptors added by hand. (See www.eyesopen.com/products/applications/ogham.html);
(iv) unless stated otherwise, reverse phase HPLC was conducted using a “Symmetry”, “NovaPak” or “Xterra” reverse phase silica column, all available from Waters Corp.;
(v) for analytical HPLC the following conditions were used:
Reverse phase analytical HPLC (Hewlett Packard Series 1100) using Waters “Symmetry” C8 column 3.5 μm; 4.6×50 mm column using 0.1% ammonium acetate/acetonitrile gradients at 2 mL/min given as % aqueous
STANDARD 75% to 5% over 3 min
FAST 45% to 5% over 2.5 min

### MEDIUM FAST 65% to 5% in 2.5 min

### SLOW 95% to 50% in 2.5 min

SUPERSLOW 100% to 80% in 2.5 min; and
(vi) the following abbreviations are used:

| | |
|---|---|
| DMF | N,N-Dimethylformamide |
| HPLC | High performance liquid chromatography |
| RPHPLC | Reverse phase high performance liquid chromatography |
| HATU | O-(7-Azabenzotriazol-1-yl)-N,N,N′,N′-tetramethyluronium |
| | hexafluorophosphate |
| THF | Tetrahydrofuran |
| DCM | Dichloromethane |
| d | Day(s) |
| h | Hour(s) |
| min | Minute(s) |
| RT | Room Temperature |

### Preparation 1

### (S)-[4-[(3,4-Dichlorophenyl)methyl]morpholin-2-yl]methanamine

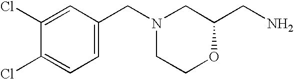

### Step 1

### (R)-2-(Benzyloxymethyl)morpholine

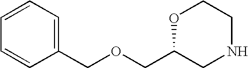

A stirred mixture of (R)-benzyl glycidyl ether (16.4 g) and 2-aminoethyl hydrogen sulfate (56.4 g) was treated with a solution of sodium hydroxide (16 g) in water (33 mL). The reaction mixture was warmed to 55° C. overnight. Water (250 mL) was added and the mixture extracted into toluene (200 mL×3). The organic layers were combined, washed with brine and then dried over magnesium sulfate, filtered and concentrated to yield the subtitle compound as a pale oil (16.5 g) which was used directly in the next step.

¹H NMR [symbol] _{(CDCl3)} 1.85-2.00 (1H, br s), 2.14-2.28 (1H, m), 2.38-2.50 (1H, m), 2.60-2.95 (3H, m), 3.39-3.55 (2H, m), 3.57-3.70 (1H, m), 3.89 (1H, dm), 4.51-4.54 (2H, ABq), 7.18-7.36 (5H, m).

### Step 2

### (R)-2-(Benzyloxymethyl)morpholine-4-carboxylic acid tert-butyl Ester

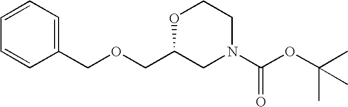

To a stirred solution of (R)-2-(benzyloxymethyl)morpholine (Step 1) (16.0 g) and triethylamine (12.9 mL) in DCM (230 mL) was added di-tert-butyl dicarbonate (18.5 g). After 16 h, aqueous sodium bicarbonate (200 mL) was added and the mixture was extracted into DCM (100 mL×3). The organic layers were combined, washed with brine and dried over magnesium sulfate, filtered and concentrated. The resulting oil was chromatographed (SiO₂: eluent 1:5 ethyl acetate/40-60 petroleum ether) to yield the subtitle compound as a colourless oil (12.1 g).

¹H NMR δ_{(CDCl3)}: 1.46 (9H, s), 2.74 (1H, t), 2.95 (1H, t), 3.42-3.65 (4H, m), 3.79-3.97 (3H, m), 4.56 (2H, s), 7.24-7.39 (5H, m).

### Step 3

### (R)-2-(Hydroxymethyl)morpholine-4-carboxylic Acid tert-butyl Ester

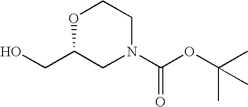

A stirred suspension of (R)-2-(benzyloxymethyl)morpholine-4-carboxylic acid tert-butyl ester (Step 2) (12.0 g) and 20% w/w palladium hydroxide on carbon (1.3 g) in ethanol (150 mL) and water (10 mL) was hydrogenated at 2 bar pressure at room temperature for 16 h. The reaction mixture was then filtered and concentrated. The resulting oil was chromatographed (SiO₂: eluent gradient 1:9 through 1:4 methanol/ethyl acetate) to yield the subtitle compound as a colourless oil (8.8 g).

¹H NMR δ_{(CDCl3)}: 1.47 (9H, s), 2.03 (1H, t), 2.70-2.81 (1H, m), 2.86-3.01 (1H, m), 3.47-3.62 (3H, m), 3.64-3.72 (1H, m), 3.77-3.96 (3H, m).

### Step 4

### (R)-2-(Methylsulfonyloxymethyl)morpholine-4-carboxylic acid tert-butyl Ester

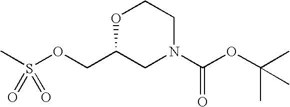

To a stirred solution of (R)-2-(hydroxymethyl)morpholine-4-carboxylic acid tert-butyl ester (Step 3) (2.17 g) and triethylamine (2.1 mL) in DCM (20 mL) was added methanesulfonyl chloride (1.0 mL) dropwise at 0° C. The reaction was allowed to warm to room temperature over 16 h. The reaction mixture was then quenched with water and the mixture was extracted into DCM (40 mL×3). The organic layers were combined and washed with water and dried over magnesium sulfate, filtered and concentrated to yield the subtitle compound as a colourless oil (3.0 g).

¹H NMR δ_{(CDCl3)} 1.47 (9H, s), 2.71-2.82 (1H, m), 2.89-3.00 (1H, m), 3.07 (3H, s), 3.55 (1H, td), 3.66-3.73 (1H, m), 3.82-3.97 (3H, m), 4.24 (2H, d).

### Step 5

### (R)-2-(Azidomethyl)morpholine-4-carboxylic Acid tert-butyl Ester

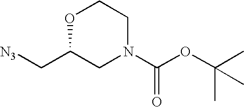

A stirred suspension of (S)-2-(methylsulfonyloxymethyl)morpholine-4-carboxylic acid tert-butyl ester (Step 4) (3.0 g), lithium iodide (0.25 g) and sodium azide (3.3 g) in DMF (12 mL) was heated at 60° C. for 20 h. The cooled reaction mixture was then quenched with water and the mixture was extracted into diethyl ether (40 mL×3). The organic layers were combined and washed with water and dried over magnesium sulfate, filtered and concentrated to yield the subtitle compound as a colourless oil (2.3 g).

¹H NMR δ_{(DMSO-d6)}: 1.40 (9H, s), 2.58-2.74 (1H, m), 2.79-2.97 (1H, m), 3.27-3.44 (3H, m), 3.44-3.56 (1H, m), 3.62-3.92 (3H, m).

### Step 6

### (S)-[4-[(3,4-Dichlorophenyl)methyl]morpholin-2-yl]methanamine

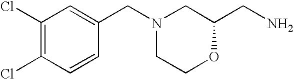

To a stirred solution of (R)-2-(azidomethyl)morpholine-4-carboxylic acid tert-butyl ester (Step 5) (2.3 g) in DCM (45 mL) at RT was added trifluoroacetic acid (6 mL). After 16 h, the reaction mixture was concentrated and dissolved in THF (13 mL). Triethylamine (1.72 mL) was added, followed by 3,4-dichlorobenzaldehyde (2.0 g), sodium triacetoxyborohydride (3.42 g), and acetic acid (0.76 mL), and the mixture was stirred at room temperature for 16 h. The reaction was then quenched with saturated sodium bicarbonate solution and the mixture was extracted into ethyl acetate (40 mL×3). The organic layers were dried over magnesium sulfate, filtered and concentrated to yield a colourless oil, which was dissolved in THF (47.5 mL) with stirring. Triphenylphosphine (2.62 g) was added and the mixture was heated at 60° C. for 40 min. Water (9.5 mL) was added and the reaction mixture was heated at reflux for a further 16 h. The mixture was concentrated and the resulting oil was chromatographed (SiO₂: eluent gradient DCM through 1:19 7 N ammonia in methanol/DCM) to yield the title compound as a colourless oil (1.8 g).

MS (ES+ve) 275/277 [M+H]⁺

Retention time: 1.16 min (STANDARD).

¹H NMR δ_{(DMSO-d6)}: 1.76 (1H, t), 2.05 (1H, td), 2.46 (1H, dd), 2.51-2.55 (2H, m), 2.59 (1H, dm), 2.73 (1H, dt), 3.28-3.35 (2H, m), 3.46 (2H, d), 3.47-3.52 (1H, m), 3.76 (1H, ddd), 7.31 (1H, dd), 7.55 (1H, d), 7.58 (1H, d).

### Preparation 2

### (R)-[4-[(3,4-Dichlorophenyl)methyl]morpholin-2-yl]methanamine

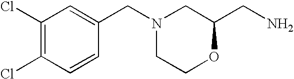

Prepared in the same fashion as (S)-[4-[(3,4-dichlorophenyl)methyl]morpholin-2-yl]methanamine (Preparation 1) starting with (S)-benzyl glycidyl ether to yield the title compound as a colourless oil.

¹H NMR δ(DMSO-d₆) 1.76 (1H, t), 2.05 (1H, td), 2.47 (1H, dd), 2.51-2.56 (2H, m), 2.59 (1H, dm), 2.73 (1H, dt), 3.28-3.36 (2H, m), 3.46 (2H, d), 3.48-3.52 (1H, m), 3.76 (1H, ddd), 7.31 (1H, dd), 7.55 (1H, d), 7.58 (1H, d).

### Preparation 3

### (S)-2-Chloro-N—[[4-[(3,4-dichlorophenyl)methyl]morpholin-2-yl]methyl]acetamide

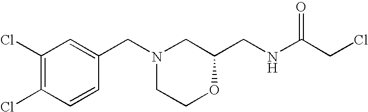

To a stirred solution of (R)-[4-[(3,4-dichlorophenyl)methyl]morpholin-2-yl]methanamine (Preparation 1) (0.55 g) and triethylamine (0.365 mL) in DCM (6 mL) at 0° C. was added chloroacetyl chloride (0.175 mL) in DCM (2 mL) dropwise. The reaction was allowed to warm to room temperature over 2 h. The reaction mixture was then quenched with water and the mixture was extracted into DCM (30 mL×3). The organic layers were combined and washed with water and dried over magnesium sulfate, filtered and concentrated to yield the subtitle compound as a colourless oil (0.52 g).

MS (ES+ve) 351/353/355 [M+H]⁺

Retention time: 1.98 min (STANDARD).

¹H NMR δ_{(CDCl3)} 1.98 (1H, t), 2.23 (1H, td), 2.71 (1H, dm), 2.77 (1H, dm), 3.23 (1H, ddd), 3.50 (2H, s), 3.51-3.57 (1H, m), 3.70 (2H, t), 3.89 (1H, ddd), 4.06 (2H, s), 6.87-6.94 (1H, m), 7.16 (1H, dd), 7.40 (1H, d), 7.43 (1H, d).

### Preparation 4

### (R)-2-Chloro-N—[[4-[(3,4-dichlorophenyl)methyl]morpholin-2-yl]methyl]acetamide

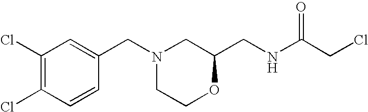

Prepared in the same way as (S)-2-chloro-N—[[4-[(3,4-dichlorophenyl)methyl]-morpholin-2-yl]methyl]acetamide (Preparation 3) from (R)-[4-[(3,4-dichlorophenyl)methyl]morpholin-2-yl]methanamine (0.412 g) to yield the title compound (0.320 g) as a colourless oil.

MS (ES+ve) 351/353/355 [M+H]⁺

Retention time: 1.98 min (STANDARD).

### Preparation 5

### (S)-2-Acetylsulfanyl-N—[[4-[(3,4-dichlorophenyl)methyl]morpholin-2-yl]methyl]acetamide

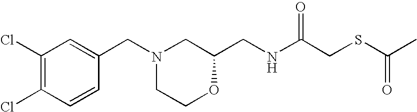

To a stirred solution of (S)-2-chloro-N—[[4-[(3,4-dichlorophenyl)methyl]morpholin-2-yl]methyl]acetamide (Preparation 3) (0.370 g) and triethylamine (0.44 mL) in DCM (10 mL) at 0° C. was added thioacetic acid (0.225 mL) dropwise. The reaction was allowed to warm to room temperature and stirred for 3 d. The reaction mixture was then quenched with aqueous sodium bicarbonate and was extracted into ethyl acetate (20 mL×3). The organic layers were combined and washed with water and dried over magnesium sulfate, filtered and concentrated to yield a brown oil which was chromatographed (SiO₂: eluent ethyl acetate) to yield the title compound as a pale brown oil (0.225 g).

MS (ES+ve) 391/393 [M+H]⁺

Retention time: 1.97 min (STANDARD).

### Preparation 6

### (S)-[4-[(3,4-Dichlorophenyl)methyl]morpholin-2-yl]methylcarbamoylmethyl-sulfanyl Sodium

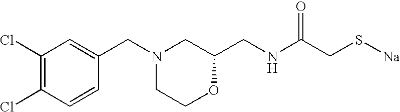

To a stirred solution of (S)-2-acetylsulfanyl-N—[[4-[(3,4-dichlorophenyl)methyl]morpholin-2-yl]methyl]acetamide (Preparation 5) (0.370 g) in methanol (5 mL) at room temperature was added sodium methoxide (1.0 M in MeOH, 0.63 mL) dropwise. The reaction was stirred for 16 h. The reaction mixture was then concentrated to yield a brown gum which was used directly where appropriate.

MS (ES+ve) 349/351 [M+H]⁺

Retention time: 1.86 min (STANDARD).

### Preparation 7

### (S)-[4-(4-Fluoro-benzyl)-morpholin-2-yl]-methylamine

2-Azidomethyl-4-(4-fluoro-benzyl)-morpholine (prepared following the method of preparation 1 as far as step 6 immediately prior to dissolution in tetrahydrofuran and addition of triphenylphosphine; 0.4 g) was dissolved in ethanol (5 mL) and 10% Palladium on C, 38 paste (0.1 g) in ethanol (3 mL) was added. The reaction mixture was hydrogenated at 3 bar pressure at room temperature. The mixture was filtered and evaporated, the residue was purified via chromatography (dichloromethane:methanol: 7N Ammonia in MeOH 75:20:5) to give the title compound (270 mg).

1H NMR δ_{(CDCL3)} 1.87 (1H, t), 2.15 (1H, td), 2.59-2.74 (4H, m), 3.41-3.56 (3H, m), 3.67 (1H, td), 3.81-3.90 (1H, m), 6.96-7.05 (2H, m), 7.24-7.31 (2H, m)

### Preparation 8

### (S)-[4-(4-Chloro-3-methyl-benzyl)-morpholin-2-yl]-methylamine

### Step 1

### (S)-(2-Azidomethyl-morpholin-4-yl)-(4-chloro-3-methyl-phenyl)-methanone

2-Azidomethyl-morpholine (prepared following the method of preparation 1 as far as step 6 immediately prior to addition of the aldehyde and triethylamine; 0.63 g), 4-chloro-3-methylbenzoic acid (0.756 g) and triethylamine (0.897 g, 1.281 ml) were dissolved in dichloromethane (6 mL) and HATU (1.853 g) was added. The reaction mixture was stirred for 1 h. The mixture was poured into water and extracted with dichloromethane thrice. The combined organic layers were washed with brine, dried and the solvents were removed to give the subtitle compound (2 g).

Retention time (standard) 2.04

MS (ES+ve) 295/297 [M+H]⁺

### Step 2

### (S)-[4-(4-Chloro-3-methyl-benzyl)-morpholin-2-yl]-methylamine

Borane-tetrahydrofuran complex (1.751 g, 20.37 ml) was added to (2-azidomethyl-morpholin-4-yl)-(4-chloro-3-methyl-phenyl)-methanone (2 g) at RT. The solution was refluxed for 1 h, then the reaction mixture was cooled to RT, MeOH was added carefully and the reaction mixture was refluxed for 1 h. The solvents were removed and the product was purified via SCX (loading in MeOH eluting with 0.07 M ammonia in methanol) and then by chromatography eluting with dichloromethane:methanol: ammonia in methanol 7 M solution (9:1:0.1 gradient to 7:3:0.3) to give the title compound (400 mg).

1H NMR δ_{(CDCL3)} 1.87 (1H, t), 2.10-2.19 (1H, m), 2.36 (3H, s), 2.62-2.75 (4H, m), 3.37-3.47 (2H, m), 3.47-3.54 (1H, m), 3.63-3.72 (1H, m), 3.83-3.90 (1H, m), 7.08 (1H, d), 7.18 (1H, s), 7.25-7.29 (1H, m).

Retention time (standard) 1.36 m/z

MS (ES+ve) 255/257 [M+H]⁺

The following compounds were made following the methods of Preparations 1, 7 or 8:

| | | | MS | Retention |
|---|---|---|---|---|
| | method | | (ES+ve) | Time |
| Compound | type | 1H NMR δ_{(CDCL3)} | (M + H)+ | (Standard) |
| (S)-[4-(3,4- | Preparation 7 | 1.86-1.95 (1H, m), | 243 | 1.07 |
| Difluoro-benzyl)- | | 2.11-2.21 (1H, m), | | |
| morpholin-2-yl]- | | 2.60-2.70 (2H, m), 2.74 (2H, d), | | |
| methylamine | | 3.38-3.48 (2H, m), | | |
| | | 3.49-3.57 (1H, m), 3.67 (1H, td), | | |
| | | 3.84-3.92 (1H, m), | | |
| | | 6.99-7.04 (1H, m), 7.04-7.13 (1H, m), | | |
| | | 7.13-7.21 (1H, m) | | |
| (S)-[4-(4-Fluoro- | Preparation 7 | 1.89 (1H, t), 2.16 (1H, td), | 239 | 1.35 |
| 2-methyl-benzyl)- | | 2.35 (3H, s), 2.56-2.77 (4H, | | |
| morpholin-2-yl]- | | m), 3.38-3.52 (3H, m), | | |
| methylamine | | 3.62 (1H, td), 3.82-3.90 (1H, m), | | |
| | | 6.77-6.90 (2H, m), | | |
| | | 7.18 (1H, dd) | | |
| (S)-[4-(4-Chloro- | Preparation 1 | 1.88 (1H, t), 2.09-2.22 (1H, | 241/243 | 1.15 |
| benzyl)- | | m), 2.60-2.73 (4H, m), | | |
| morpholin-2-yl]- | | 3.40-3.53 (3H, m), | | |
| methylamine | | 3.60-3.72 (1H, m), 3.82-3.92 (1H, m), | | |
| | | 7.22-7.32 (4H, m) | | |
| (S)-[4-(3,4- | Preparation 8 | | 289/291 | 1.89 |
| Dichloro-2- | | | | |
| methyl-benzyl)- | | | | |
| morpholin-2-yl]- | | | | |
| methylamine) | | | | |

### Preparation 9

### 4-Carboxymethylsulfanyl-3-chloro-benzoic acid tert-butyl Ester

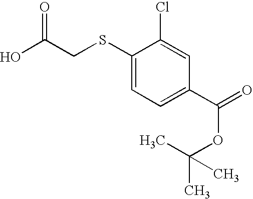

### Step 1

### 3-Chloro-4-methoxycarbonylmethylsulfanyl-benzoic acid tert-butyl Ester

3-Chloro-4-fluoro-benzoic acid tert-butyl ester (1.3 g) and potassium carbonate (0.857 g) were slurried in DMF (3 mL). Mercapto-acetic acid methyl ester (0.659 g, 0.555 ml) was added. The mixture was stirred for 1 h and was then diluted with sodium bicarbonate solution and extracted with ethyl acetate. The extracts were washed with brine, dried over MgSO₄, filtered and evaporated to give the subtitle compound (1.3 g).

Retention Time (standard) 2.72

MS 315 [M−H]⁻ (ES−)

### Step 2

### 4-Carboxymethylsulfanyl-3-chloro-benzoic acid tert-butyl Ester

3-Chloro-4-methoxycarbonylmethylsulfanyl-benzoic acid tert-butyl ester (1.3 g) was dissolved in tetrahydrofuran (30 ml) and sodium hydroxide solution (0.1968 g, 4.92 ml) was added. The mixture was stirred for 2 h and was then acidified with AcOH. The solution was extracted using dichloromethane, evaporated and then azeotroped with toluene to give the title compound (1.0 g).

Retention Time (standard) 1.26

MS 301 [M−H]⁻ (ES−)

1H NMR δ_{(CDCL3)} 1.58 (9H, s), 3.79 (2H, s), 7.33 (1H, d), 7.84 (1H, dd), 7.95 (1H, d).

### Preparation 10

### 4-Carboxymethylsulfanyl-3-fluoro-benzoic acid tert-butyl Ester

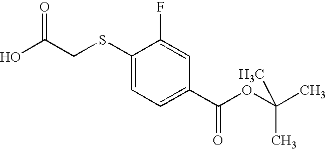

This compound was prepared from 3,4-difluorobenzoic acid tert-butyl ester following the method of Preparation 9.

Retention Time (standard) 0.56.

MS 243 [M−H]⁻ (ES−)

1H NMR δ_{(CDCL3)} 3.88 (2H, s), 3.92 (3H, s), 7.52 (1H, t), 7.69 (1H, d), 7.81 (1H, d).

### Preparation 11

### 6-Carboxymethylsulfanyl-nicotinic Acid Methyl Ester

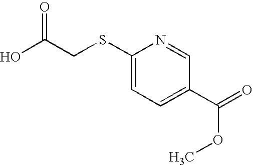

### Step 1

### 6-tert-Butoxycarbonylmethylsulfanyl-nicotinic Acid Methyl Ester

Bromo-acetic acid tert-butyl ester (1.153 g), 6-mercapto-nicotinic acid methyl ester (1 g) and potassium carbonate (0.817 g) were combined in DMF and stirred at RT overnight. The solvent was evaporated. The residue was redissolved in dichloromethane, washed with water, dried, filtered and evaporated. The residue was purified by chromatography (95:5 isohexane:ethyl acetate) to give the subtitle compound.

Retention Time (standard) 2.33

MS 228 [M+H]+ (E+)

### Step 2

### 6-Carboxymethylsulfanyl-nicotinic Acid Methyl Ester

6-tert-Butoxycarbonylmethylsulfanyl-nicotinic acid methyl ester was dissolved in DCM (6 mL) and trifluoroacetic acid (3 mL) was added. The solution was stirred at RT overnight. Evaporation of the volatiles gave the title compound (700 mg).

Retention Time (standard) 0.44

MS 228 [+H]+ (ES+)

1H NMR δ_{(CDCL3)} 3.83 (2H, s), 3.98 (3H, s), 7.46 (1H, dd), 8.23 (1H, dd), 9.04 (1H, t).

### Preparation 12

### 4-Carboxymethylsulfanyl-benzoic Acid Methyl Ester

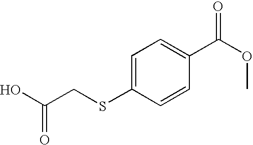

This compound was prepared using 4-mercaptobenzoic acid by the method of Preparation 11.

Retention Time (standard) 0.45

MS 227 [M+H]+ (ES+)

1H NMR δ_{(DMSO)} 3.83 (3H, s), 3.95 (2H, s), 7.41 (2H, d), 7.87 (2H, d)

### Preparation 13

### 2-Amino-6-carboxymethylsulfanyl-nicotinic Acid Methyl Ester

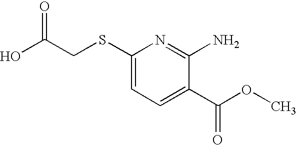

### Step 1

### 2-Amino-6-tert-butoxycarbonylmethylsulfanyl-nicotinic Acid Methyl Ester

Mercapto-acetic acid tert-butyl ester (0.37 g) was dissolved in DMF (5 mL) and potassium tert-butoxide (0.280 g) was added. The reaction mixture was stirred for 30 min. 2-Amino-6-chloro-nicotinic acid methyl ester (0.466 g) was added and the reaction was stirred overnight. The mixture was poured into water and was extracted with EtOAc. The extracts were washed with brine, dried over MgSO₄, filtered and evaporated. The residue was purified by chromatography (isohexane:EtOAc 9:1) to give the subtitle compound (480 mg).

Retention Time (standard) 2.39

MS 299 [M+H]+ (ES+)

### Step 2

### 2-Amino-6-carboxymethylsulfanyl-nicotinic Acid Methyl Ester

2-Amino-6-tert-butoxycarbonylmethylsulfanyl-nicotinic acid methyl ester was taken up in 6N HCl solution and stirred at RT for 72 h. The mixture was evaporated to dryness to give the title compound (360 mg).

Retention Time (standard) 0.45

MS 243 [M+H]+ (ES+)

1H NMR δ_{(CD3OD)} 2.35 (3H, s), 2.49 (2H, s), 5.08 (1H, d), 6.49 (1H, d).

The following compounds were prepared using the method of Preparation 13 from the appropriately substituted esters:

| | | Retention | MS | |
|---|---|---|---|---|
| | | Time | [M − H]⁻ | |
| Compound | Starting ester | (standard) | (ES−) | 1H NMR δ_{(CD3OD)} |
| 2-Amino-6- | 2-Amino-5-fluoro- | 0.50 | 259 | 3.87 (3H, s), |
| carboxymethylsulfanyl- | 6-(toluene-4- | | | 4.03 (2H, s), 7.72 (1H, |
| 5-fluoro-nicotinic acid | sulfonyl)-nicotinic | | | d) |
| methyl ester | acid methyl ester | | | |
| 4- | 3,4-Difluoro- | 0.56 | 243 | 3.88 (2H, s), |
| Carboxymethylsulfanyl- | benzoic acid | | | 3.92 (3H, s), 7.52 (1H, |
| 3-fluoro-benzoic acid | methyl ester | | | t), 7.69 (1H, d), |
| methyl ester | | | | 7.81 (1H, d) |

### Preparation 14

### 2-Amino-6-carboxymethylsulfanyl-5-fluoro-nicotinic Acid Methyl Ester

### Step a

### 2-Chloro-5-fluoro-6-[(4-methylphenyl)thio]nicotinic Acid

Lithium hydroxide monohydrdrate (1.8 g) was added to a solution of ethyl 2-chloro-5-fluoro-6-[(4-methylphenyl)thio]nicotinate (7.0 g) in THF (100 mL). Water (20 mL) was added and the solution stirred vigorously for 18 h. The mixture was diluted with water (400 mL) and washed with ether. The aqueous was acidified with acetic acid and extracted with ether. The ether was dried and evaporated to give the subtitle compound (6.0 g).

¹H NMR δ_{(DMSO)} 2.37 (3H, s), 7.31 (2H, d), 7.47 (2H, d), 8.12 (1H, d).

### Step b

### 2-Amino-5-fluoro-6-[(4-methylphenyl)thio]nicotinic Acid

The product from step a) (1 g) and aqueous ammonia (density 0.880) (20 mL) were heated in a sealed tube at 140° C. for 5 h. The cooled mixture was evaporated and the residue was coevaporated with methanol (×3) then dissolved in methanol and acidified with acetic acid. The mixture was evaporated and coevaporated with methanol (×2) then toluene (×2). Final drying under high vacuum gave the subtitle compound (0.95 g).

¹H NMR δ_{(DMSO)} 2.33 (3H, s), 7.24 (3H, m), 7.38 (2H, d).

### Step c

### 2-Amino-5-fluoro-6-[(4-methylphenyl)thio]nicotinic Acid Methyl Ester

The product from step b) (0.95 g) was stirred in thionyl chloride (5 mL) and heated under reflux for 1 h. The solvent was evaporated and the residue dissolved in ice cooled methanol (10 mL). The mixture was evaporated and the residue was mixed with saturated sodium bicarbonate solution and extracted with ethyl acetate. The extracts were washed with brine then dried and evaporated. Purification by flash chromatography (ethyl acetate/isohexane 9:1) gave the subtitle compound (0.3 g).

¹H NMR δ_{(CDCl3)} 2.39 (3H, s), 3.85 (3H, s), 7.21 (2H, d), 7.42 (2H, d), 7.67 (1H, d).

### Step d

### 2-Amino-5-fluoro-6-[(4-methylphenyl)sulfonyl]nicotinic Acid Methyl Ester

The product from step c) (0.74 g) was stirred in dichloromethane (5 mL) and m-chloroperoxybenzoic acid (1.13 g of 77%) was added. The mixture was stirred for 2 h then was washed with sodium bicarbonate solution followed by sodium metabisulfite solution then brine. The solution was dried and evaporated and the residue purified by flash chromatography (ethyl acetate/dichloromethane, 1:1) to give the title compound (0.43 g).

¹H NMR δ_{(CDCl3)} 2.44 (3H, s), 3.90 (3H, s), 7.35 (2H, m), 7.94 (3H, m).

MS 325 [M+H]⁺ (APCI+).

### EXAMPLE 1

### (S)-6-[[4-[(3,4-Dichlorophenyl)methyl]morpholin-2-yl]methylcarbamoylmethyl-sulfanyl]nicotinic Acid

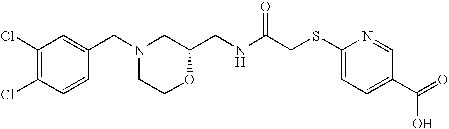

A stirred solution of (S)-2-chloro-N—[[4-[(3,4-dichlorophenyl)methyl]morpholin-2-yl]methyl]acetamide (Preparation 3) (0.141 g), sodium acetate (0.066 g) and 6-mercaptonicotinic acid (0.078 g) in ethanol (10 mL) at 0° C. was heated at reflux for 24 h. The reaction mixture was concentrated and redissolved in water (3 mL), acetonitrile (3 mL) and acetic acid (10 drops), filtered through a Whatman Puradisc polypropylene 0.45 μM filter and subjected to purification by RPHPLC (Novapak, 0.1% ammonium acetate/acetonitrile) to afford the title compound as a colourless solid (0.079 g).

MS (ES-ve) 468/470 [M−H]⁻

Retention time: 1.07 min (STANDARD).

¹H NMR δ_{(CD3OD)}: 1.83 (1H, t), 1.93-2.05 (1H, m), 2.58 (1H, dm), 2.65 (1H, dm), 3.29 (2H, dd), 3.43 (2H, s), 3.50-3.61 (2H, m), 3.79 (1H, dm), 3.90 (2H, ABq), 7.24 (1H, dd), 7.37 (1H, dd), 7.45-7.50 (2H, m), 8.14 (1H, dd), 9.01 (1H, dd).

EXAMPLES 2-6 were prepared analogously to EXAMPLE 1 using the appropriate thiol and the appropriate enantiomer of the chloroacetamide—see Table below.

### EXAMPLE 7

### (S)-4-[[4-[(3,4-Dichlorophenyl)methyl]morpholin-2-yl]methylcarbamoyl-methoxy]benzoic Acid Methyl Ester

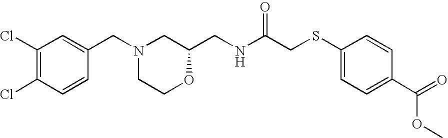

To a stirred solution of (S)-[4-[(3,4-dichlorophenyl)methyl]morpholin-2-yl]methanamine (Preparation 1) (0.141 g), diisopropylethylamine (0.130 mL) and 2-(4-methoxycarbonylphenoxy)acetic acid (0.084 g) in N-methylpyrrolidinone (10 mL) at 0° C. was added HATU (0.152 g). The reaction mixture was allowed to warm to room temperature and stirred for 16 h. The reaction mixture was then quenched with aqueous sodium bicarbonate and was extracted into 1:1 ethyl acetate/diethyl ether (20 mL×3). The organic layers were combined and washed with water and dried over magnesium sulfate, filtered and concentrated to yield a pale oil which was chromatographed (SiO₂: eluent gradient DCM to 2:98 methanol/DCM) to yield the subtitle compound as a pale gum (0.140 g).

MS (ES+ve) 467/469 [M+H]⁺

Retention time: 2.27 min (STANDARD).

### EXAMPLE 7A

### (S)-4-[[4-[(3,4-Dichlorophenyl)methyl]morpholin-2-yl]methylcarbamoylmethoxy]-benzoic Acid

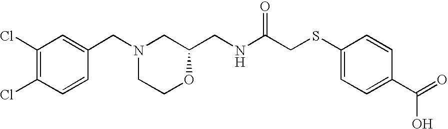

A solution of (S)-4-[[4-[(3,4-dichlorophenyl)methyl]morpholin-2-yl]methylcarbamoylmethoxy]benzoic acid methyl ester (Example 7) (0.130 g), in 1:1:1 methanol/water/THF (3 mL) at room temperature was stirred with lithium hydroxide monohydrate (0.118 g) for 16 h. The reaction mixture was concentrated and redissolved in water (3 mL), acetonitrile (3 mL) and acetic acid (10 drops), filtered through a Whatman Puradisc polypropylene 0.45 μM filter and subjected to purification by RPHPLC (Novapak, 0.1% ammonium acetate/acetonitrile) to afford the title compound as a white solid (0.034 g).

MS (ES-ve) 451/453 [M−H]⁻

Retention time: 1.12 min (STANDARD).

¹H NMR δ_{(CD3OD)}: 1.75 (1H, t), 2.00 (1H, td), 2.50 (1H, d), 2.59 (1H, d), 3.21-3.25 (2H, m), 3.35 (2H, s), 3.45-3.57 (2H, m), 3.73 (1H, dm), 4.47 (2H, s), 6.87 (2H, dm), 7.16 (1H, dd), 7.36 (1H, d), 7.41 (1H, d), 7.85 (2H, dm).

### EXAMPLE 8

### (S)-4-[[4-[(3,4-Dichlorophenyl)methyl]morpholin-2-yl]methylcarbamoylmethyl-sulfanyl]-3-(trifluoromethyl)benzoic Acid Methyl Ester

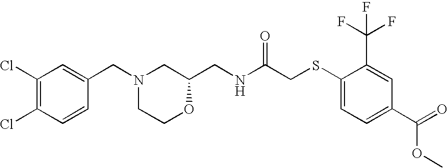

A solution of (S)-[4-[(3,4-dichlorophenyl)methyl]morpholin-2-yl]methylcarbamoylmethylsulfanylsodium (Preparation 6) (0.104 g) in DMF (1.0 mL) was added to a stirred solution of methyl-4-fluoro-3-(trifluoromethyl)benzoate (0.080 g) in DMF (0.5 mL) at room temperature and stirred for 16 h. The reaction mixture was then quenched with aqueous sodium bicarbonate and was extracted into DCM (10 mL×3). The organic layers were combined and washed with water and dried over magnesium sulfate, filtered and concentrated to yield a pale oil which was chromatographed (SiO₂: eluent gradient DCM to 5:95 methanol/DCM) to yield the subtitle compound as a colourless oil (0.090 g).

MS (ES+ve) 551/553 [M+H]⁺; Retention time: 2.69 min (STANDARD).

TABLE 1

| | | | MS | |
|---|---|---|---|---|
| | | | (ES−ve) | Retention time |
| Example | Name | ¹H NMR δ_{(CD3OD)} | (M − H)− | (Standard) |
| 2 | (S)-4-[[4-[(3,4- | 1.76 (1H, t), 2.05 (1H, td), 2.60 (2H, t), 3.27 (2H, d), | 467/469 | 1.18 |
| | dichlorophenyl)methyl]morpholin-2- | 3.39 (2H, s), 3.49-3.61 (2H, m), 3.73 (2H, s), 3.81 (1H, dm), | | |
| | yl]methylcarbamoylmethylsulfanyl]- | 7.25 (1H, dd), 7.39 (2H, d), 7.46 (1H, d), 7.50 (1H, d), | | |
| | benzoic acid | 7.94 (2H, d) | | |
| 3 | (R)-6-[[4-[(3,4- | 1.77 (1H, t), 1.90-1.98 (1H, m), 2.53 (1H, dm), 2.60 (1H, | 468/470 | 1.17 |
| | dichlorophenyl)methyl]morpholin-2- | dm), 3.26 (2H, t), 3.38 (2H, s), 3.48-3.56 (2H, m), | | |
| | yl]methylcarbamoylmethylsulfanyl]- | 3.76 (1H, dm), 3.85 (2H, d), 7.21 (1H, dd), 7.29 (1H, dd), | | |
| | nicotinic acid | 7.43-7.46 (2H, m), 8.10 (1H, dd), 8.97 (1H, dd). | | |
| 4 | (R)-4-[[4-[(3,4- | 1.74 (1H, t), 2.03 (1H, td), 2.57 (2H, app t), 3.24 (2H, dd), | 467/469 | 1.26 |
| | dichlorophenyl)methyl]morpholin-2- | 3.37 (2H, s), 3.48-3.58 (2H, m), 3.70 (2H, s), 3.78 (1H, | | |
| | yl]methylcarbamoylmethylsulfanyl]- | dm), 7.22 (1H, dd), 7.37 (2H, dm), 7.44 (1H, d), 7.47 (1H, | | |
| | benzoic acid | d), 7.92 (2H, dm). | | |
| 5 | (S)-4-[[4-[(3,4- | 1.30-1.38 (2H, m), 1.56 (9H, s), 1.69-1.74 (1H, m), | MS | 1.66 |
| | dichlorophenyl)methyl]morpholin-2- | 2.58 (1H, tm), 3.25-3.28 (2H, m), 3.39 (2H, s), 3.50-3.60 (2H, | (ES+ve) | |
| | yl]methylcarbamoylmethylsulfanyl]-3- | m), 3.73 (2H, s), 3.80 (1H, d), 7.26 (1H, dd), 7.42 (1H, d), | 525/ | |
| | tert-butyl-benzoic acid | 7.46 (1H, d), 7.51 (1H, d), 7.78 (1H, dd), 8.12 (1H, d). | 527 (M + H)+ | |
| 6 | (S)-3-[[4-[(3,4- | 1.74 (1H, app tm), 2.03 (1H, td), 2.51-2.58 (2H, m), | MS | 1.24 |
| | dichlorophenyl)methyl]morpholin-2- | 3.20 (2H, d), 3.38 (2H, s), 3.48-3.56 (2H, m), 3.65 (2H, s), | (ES+ve) | |
| | yl]methylcarbamoylmethylsulfanyl]- | 3.76 (1H, dm), 7.23 (1H, dd), 7.31 (1H, t), 7.40-7.51 (3H, | 469/ | |
| | benzoic acid | m), 7.80 (1H, dm), 7.97-7.99 (1H, m). | 471 (M + H)+ | |

TABLE 2
The following compounds were prepared analogously to Example 7A from the appropriate esters:

| | | | MS | Retention |
|---|---|---|---|---|
| | | | (ES−ve) | time |
| Example | Name | ¹H NMR δ_{(CD3OD)} | (M − H)− | (Standard) |
| 8A | (S)-4-[[4-[(3,4- | 1.77 (1H, t), 2.05 (1H, td), 2.58 (1H, d), 2.62 (1H, d), | 535/537 | 1.41 |
| | dichlorophenyl)methyl]morpholin-2- | 3.25 (2H, d), 3.39 (2H, d), 3.50-3.58 (2H, m), 3.78 (1H, dm), | | |
| | yl]methylcarbamoylmethylsulfanyl]- | 3.82 (2H, s), 7.21 (1H, dd), 7.43 (1H, d), 7.47 (1H, d), | | |
| | 3-(trifluoromethyl)benzoic acid | 7.65 (1H, d), 8.13 (1H, d), 8.27 (1H, d). | | |
| 9A | (S)-5-chloro-6-[[4-[(3,4- | 1.77 (1H, t), 1.89-1.95 (1H, m), 2.52 (1H, dm), 2.60 (1H, | 502/504 | 1.24 |
| | dichlorophenyl)methyl]morpholin-2- | dm), 3.25 (2H, t), 3.37 (2H, s), 3.45-3.50 (1H, m), | | |
| | yl]methylcarbamoylmethylsulfanyl]- | 3.49-3.56 (1H, m), 3.75 (1H, dm), 3.90 (2H, ABq), 7.21 (1H, | | |
| | nicotinic acid | dd), 7.43 (1H, d), 7.45 (1H, d), 8.14 (1H, d), 8.90 (1H, d) | | |

TABLE 3
The following compound was prepared following the method
of Example 8 using the appropriate aryl fluoride:

| | | MS | Retention |
|---|---|---|---|
| | | (ES+ve) | time |
| Example | Name | (M + H)+ | (Standard) |
| 9 | (S)-5-chloro-6-[[4-[(3,4- | 518/520 | 2.52 |
| | dichlorophenyl)methyl]morpholin-2- | | |
| | yl]methylcarbamoylmethylsulfanyl]- | | |
| | nicotinic acid methyl ester | | |

### EXAMPLE 10

### 2-Amino-6-({[4-(3,4-dichloro-2-methyl-benzyl)-morpholin-2-ylmethyl]-carbamoyl}-methylsulfanyl)-5-fluoro-nicotinic Acid Methyl Ester

(S)-[4-(3,4-Dichloro-2-methyl-benzyl)-morpholin-2-yl]-methylamine (0.18 g), 2-amino-6-carboxymethylsulfanyl-5-fluoro-nicotinic acid methyl ester (0.234 g) and triethylamine (0.180 ml) were combined in dichloromethane (4 mL). HATU (0.260 g) was added. The reaction mixture was stirred at RT for 1 h. The reaction mixture was poured into water, extracted with dichloromethane and the solvents were evaporated. The product was purified via SCX resin eluting with ammonia in methanol solution (0.7 M) to give the title compound (205 mg).

Retention Time (standard) 2.74

MS (ES+ve) 531/533 [M+H]⁺

Examples 11-28 & 30-33 were prepared from the appropriate acids and amines following the method of Example 10. (Table 4)

The following compound was prepared following the method of Example 8 using the appropriate aryl chloride:

| | | MS | Retention |
|---|---|---|---|
| | | (ES+ve) | time |
| Example | Name | (M + H)+ | (Standard) |
| 29 | 2-Amino-6-({[4-(3,4-dichloro- | 499/501 | 2.39 |
| | benzyl)-morpholin-2-ylmethyl]- | | |
| | carbamoyl}-methylsulfanyl)-5-fluoro- | | |
| | nicotinic acid methyl ester | | |

TABLE 4

| | | Retention Time | MS (ES+ve) |
|---|---|---|---|
| Example | Name | (standard) | (M + H)+ |
| 11 | 4-[[(2S)-4-[(3,4-dichloro-2-methyl-phenyl)methyl]morpholin-2- | 2.49 | 526/528 |
| | yl]methylcarbamoylmethylsulfanyl]pyridine-2-carboxylic acid isopropyl ester | | |
| 12 | 4-[[(2S)-4-[(3,4-dichloro-2-methyl-phenyl)methyl]morpholin-2- | 2.74 | 515/517 |
| | yl]methylcarbamoylmethylsulfanyl]-3-fluoro-benzoic acid methyl ester | | |
| 13 | 2-amino-6-[[(2S)-4-[(3,4-dichlorophenyl)methyl]morpholin-2- | 2.49 | 517/519 |
| | yl]methylcarbamoylmethylsulfanyl]-5-fluoro-pyridine-3-carboxylic acid methyl ester | | |
| 14 | 2-amino-6-[[(2S)-4-[(4-chlorophenyl)methyl]morpholin-2- | 2.17 | 465/467 |
| | yl]methylcarbamoylmethylsulfanyl]pyridine-3-carboxylic acid methyl ester | | |
| 15 | 4-[[(2S)-4-[(4-chlorophenyl)methyl]morpholin-2- | 2.00 | 478/480 |
| | yl]methylcarbamoylmethylsulfanyl]pyridine-2-carboxylic acid isopropyl ester | | |
| 16 | 4-[[(2S)-4-[(4-chloro-3-methyl-phenyl)methyl]morpholin-2- | 2.44 | 481/483 |
| | yl]methylcarbamoylmethylsulfanyl]-3-fluoro-benzoic acid methyl ester | | |
| 17 | 4-[[(2S)-4-[(3,4-dichlorophenyl)methyl]morpholine-2-yl]methylcarbamoylmethylsulfanyl]- | 2.49 | 501/503 |
| | 3-fluoro-benzoic acid methyl ester | | |
| 18 | 2-amino-6-[[(2S)-4-[(3,4-difluorophenyl)methyl]morpholin-2- | 2.09 | 467 |
| | yl]methylcarbamoylmethylsulfanyl]pyridine-3-carboxylic acid methyl ester | | |
| 19 | 2-amino-6-[[(2S)-4-[(4-chloro-3-methyl-phenyl)methyl]morpholin-2- | 2.45 | 497/499 |
| | yl]methylcarbamoylmethylsulfanyl]-5-fluoro-pyridine-3-carboxylic acid methyl ester | | |
| 20 | 2-amino-6-[[(2S)-4-[(4-chlorophenyl)methyl]morpholin-2- | 2.27 | 483/485 |
| | yl]methylcarbamoylmethylsulfanyl]-5-fluoro-pyridine-3-carboxylic acid methyl ester | | |
| 21 | 4-[[(2S)-4-[(4-fluoro-2-methyl-phenyl)methyl]morpholin-2- | 2.05 | 476 |
| | yl]methylcarbamoylmethylsulfanyl]pyridine-2-carboxylic acid isopropyl ester | | |
| 22 | 2-amino-5-fluoro-6-[[(2S)-4-[(4-fluoro-2-methyl-phenyl)methyl]morpholin-2- | 2.03 | 481 |
| | yl]methylcarbamoylmethylsulfanyl]pyridine-3-carboxylic acid methyl ester | | |
| 23 | 2-[[(2S)-4-[(4-fluorophenyl)methyl]morpholin-2- | 2.09 | 482 |
| | yl]methylcarbamoylmethylsulfanyl]quinoline-6-carboxylic acid methyl ester | | |
| 24 | 2-amino-6-[[(2S)-4-[(4-fluoro-2-methyl-phenyl)methyl]morpholin-2- | 2.27 | 461 (ES−) |
| | yl]methylcarbamoylmethylsulfanyl]pyridine-3-carboxylic acid methyl ester | | |
| 25 | 6-[[(2S)-4-[(4-fluoro-2-methyl-phenyl)methyl]morpholin-2- | 2.13 | 448 |
| | yl]methylcarbamoylmethylsulfanyl]pyridine-3-carboxylic acid methyl ester | | |
| 26 | 2-amino-6-[[(2S)-4-[(4-fluorophenyl)methyl]morpholin-2- | 1.98 | 447 |
| | yl]methylcarbamoylmethylsulfanyl]pyridine-3-carboxylic acid methyl ester | | |
| 27 | 6-[[(2S)-4-[(4-fluorophenyl)methyl]morpholin-2- | 1.66 | 434 |
| | yl]methylcarbamoylmethylsulfanyl]pyridine-3-carboxylic acid methyl ester | | |
| 28 | 6-[[(2S)-4-[(3,4-difluorophenyl)methyl]morpholin-2- | 1.98 | 452 |
| | yl]methylcarbamoylmethylsulfanyl]pyridine-3-carboxylic acid methyl ester | | |
| 30 | 4-[[(2S)-4-[(4-chlorophenyl)methyl]morpholin-2-yl]methylcarbamoylmethylsulfanyl]-3- | 2.74 | 509/511 |
| | fluoro-benzoic acid tert-butyl ester | | |
| 31 | 3-fluoro-4-[[(2S)-4-[(4-fluoro-2-methyl-phenyl)methyl]morpholin-2- | 2.84 | 507 |
| | yl]methylcarbamoylmethylsulfanyl]benzoic acid tert-butyl ester | | |
| 32 | 4-[[(2S)-4-[(3,4-difluorophenyl)methyl]morpholin-2-yl]methylcarbamoylmethylsulfanyl]- | 2.67 | 511 |
| | 3-fluoro-benzoic acid tert-butyl ester | | |
| 33 | 4-[[(2S)-4-[(4-fluorophenyl)methyl]morpholin-2-yl]methylcarbamoylmethylsulfanyl]-3- | 2.60 | 493 |
| | fluoro-benzoic acid tert-butyl ester | | |

### EXAMPLE 10A

### 2-Amino-6-({[4-(3,4-dichloro-2-methyl-benzyl)-morpholin-2-ylmethyl]-carbamoyl}-methylsulfanyl)-5-fluoro-nicotinic Acid

2-Amino-6-({[4-(3,4-dichloro-2-methyl-benzyl)-morpholin-2-ylmethyl]-carbamoyl}-methylsulfanyl)-5-fluoro-nicotinic acid methyl ester (205 mg) was dissolved in THF (2 mL) and LiOH (0.05 g) in water (2 mL) was added. The reaction mixture was stirred at RT overnight. The reaction mixture was evaporated, redissolved in MeOH/AcOH and purified via RP-prep-HPLC 95:50 over 30 mins to give the title compound (0.186 g).

Retention Time (standard) 1.58

MS (ES+ve) 517/519 [M+H]⁺

1H NMR δ_{(CD3OD+NaOD)} 1.76 (1H, t), 1.85-1.96 (1H, m), 2.38 (3H, s), 2.40-2.48 (1H, m), 2.49-2.57 (1H, m), 3.14-3.27 (2H, m), 3.32 (4H, s), 3.40-3.53 (2H, m), 3.67-3.73 (1H, m), 7.09 (1H, d), 7.25 (1H, d), 7.74 (1H, d)

Examples 11A-29A were prepared from the appropriate esters following the method of Example 10A. (Table 5)

### EXAMPLE 30A

### (S)-4-({[4-(4-Chloro-benzyl)-morpholin-2-ylmethyl]-carbamoyl}-methylsulfanyl)-3-fluoro-benzoic Acid

(S)-4-({[4-(4-Chloro-benzyl)-morpholin-2-ylmethyl]-carbamoyl}-methylsulfanyl)-3-fluoro-benzoic acid tert-butyl ester (from (S)-[4-(4-chloro-benzyl)-morpholin-2-yl]-methylamine (0.0395 g) and 4-carboxymethylsulfanyl-3-fluoro-benzoic acid tert-butyl ester (0.047 g)) was dissolved in dichloromethane (4 mL) and trifluoroacetic acid (4 mL). The reaction mixture was stirred at RT for 4 h. The solvent was evaporated, the residue was redissolved in MeOH and purified via RP-prep-HPLC (ammonium acetate:acetonitrile; 95:50 over 30 min) to give the title compound (0.015 g).

Retention Time (standard) 1.02

MS (ES-ve) 451/453 [M−H]⁻

1H NMR δ_{(CD3OD)} 1.80-1.91 (1H, m), 2.06-2.17 (1H, m), 2.68 (2H, t), 3.22-3.28 (2H, m), 3.44-3.62 (4H, m), 3.73-3.86 (3H, m), 7.27-7.38 (4H, m), 7.45-7.52 (1H, m), 7.70 (1H, d), 7.81 (1H, d).

### EXAMPLE 31A

### 3-Fluoro-4-[[(2S)-4-[(4-fluoro-2-methyl-phenyl)methyl]morpholin-2-yl]methylcarbamoylmethylsulfanyl]benzoic Acid

3-Fluoro-4-[[(2S)-4-[(4-fluoro-2-methyl-phenyl)methyl]morpholin-2-yl]methylcarbamoylmethylsulfanyl]benzoic acid tert-butyl ester (from (S)-[4-(4-fluoro-2-methyl-benzyl)-morpholin-2-yl]-methylamine (0.05 g), and 4-carboxymethylsulfanyl-3-fluoro-benzoic acid tert-butyl ester (0.0661 g)) was dissolved in 6 M HCl aq. and stirred for 2 h. The reaction mixture was evaporated, the residue was redissolved in MeOH and purified via RP-prep-HPLC (ammonium acetate:acetonitrile; 95:50 over 30 min) to give the title compound (32 mg).

Retention Time (standard) 1.10

MS 449/450 [M−H]⁻ (ES−)

1H NMR δ_{(CD3OD+NaOD)} 1.82-1.90 (1H, m), 2.10-2.18 (1H, m), 2.34 (3H, s), 2.59-2.70 (2H, m), 3.21-3.26 (2H, m), 3.42-3.54 (4H, m), 3.73 (2H, s), 3.76-3.82 (1H, m), 6.79-6.91 (2H, m), 7.19 (1H, dd), 7.47 (1H, t), 7.67 (1H, dd), 7.78 (1H, dd).

Examples 31A & 32A were prepared from the appropriate esters following the method of Example 30A.

TABLE 5

| Ex- | | | Retention | MS |
|---|---|---|---|---|
| am- | | | Time | (ES+ve) |
| ple | Name | 1H NMR δ_{(CD3OD+NaOD)} | (standard) | (M + H)+ |
| 11A | 4-[[(2S)-4-[(3,4-dichloro-2-methyl- | 1.75-1.84 (1H, m), 2.01-2.11 (1H, m), 2.42 (3H, s), | 1.41 | 484/486 |
| | phenyl)methyl]morpholin-2- | 2.47-2.54 (1H, m), 2.55-2.63 (1H, m), 3.21-3.26 (2H, m), 3.40 (2H, | | |
| | yl]methylcarbamoylmethylsulfanyl]pyridine- | s), 3.43-3.52 (2H, m), 3.71-3.78 (1H, m), 3.81 (2H, d), | | |
| | 2-carboxylic acid | 7.13 (1H, d), 7.27 (1H, d), 7.31-7.35 (1H, m), 7.92 (1H, s), | | |
| | | 8.35 (1H, d) | | |
| 12A | 4-[[(2S)-4-[(3,4-dichloro-2-methyl- | 1.71-1.81 (1H, m), 2.00-2.10 (1H, m), 2.42 (3H, s), | 1.56 | 501/503 |
| | phenyl)methyl]morpholin-2- | 2.48-2.61 (2H, m), 3.21 (2H, d), 3.39 (2H, s), 3.42-3.52 (2H, m), | | |
| | yl]methylcarbamoylmethylsulfanyl]- | 3.68 (2H, s), 3.71-3.79 (1H, m), 7.12 (1H, d), 7.27 (1H, d), | | |
| | 3-fluoro-benzoic acid | 7.42 (1H, t), 7.64 (1H, dd), 7.75 (1H, dd) | | |
| 13A | 2-amino-6-[[(2S)-4-[(3,4- | 1.68-1.77 (1H, m), 1.83-1.92 (1H, m), 2.52 (2H, dd), | 1.25 | 503/505 |
| | dichlorophenyl)methyl]morpholin-2- | 3.17-3.26 (1H, m), 3.32 (4H, s), 3.43-3.58 (2H, m), 3.64-3.85 (2H, | | |
| | yl]methylcarbamoylmethylsulfanyl]- | m), 7.18 (1H, dd), 7.41 (1H, d), 7.43 (1H, d), 7.74 (1H, d) | | |
| | 5-fluoro-pyridine-3-carboxylic acid | | | |
| 14A | 2-amino-6-[[(2S)-4-[(4- | 1.76 (1H, t), 1.85-1.94 (1H, m), 2.47-2.53 (1H, m), | 0.95 | 449/451 |
| | chlorophenyl)methyl]morpholin-2- | 2.55-2.61 (1H, m), 3.17-3.25 (2H, m), 3.31-3.39 (2H, m), | | |
| | yl]methylcarbamoylmethylsulfanyl]pyridine- | 3.45-3.57 (2H, m), 3.63-3.75 (2H, m), 3.82 (1H, d), 6.48 (1H, d), | | |
| | 3-carboxylic acid | 7.20-7.29 (4H, m), 7.94 (1H, d) | | |
| 15A | 4-[[(2S)-4-[(4- | 1.79-1.88 (1H, m), 2.05-2.15 (1H, m), 2.57-2.71 (2H, m), | 1.15 | 436/438 |
| | chlorophenyl)methyl]morpholin-2- | 3.21-3.26 (2H, m), 3.45 (2H, s), 3.47-3.60 (2H, m), | | |
| | yl]methylcarbamoylmethylsulfanyl]pyridine- | 3.73-3.85 (3H, m), 7.29 (4H, s), 7.32-7.38 (1H, m), 7.92 (1H, s), | | |
| | 2-carboxylic acid | 8.32-8.39 (1H, m) | | |
| 16A | 4-[[(2S)-4-[(4-chloro-3-methyl- | 1.77-1.89 (1H, m), 2.07-2.19 (1H, m), 2.36 (3H, d), | 1.20 | 467/469 |
| | phenyl)methyl]morpholin-2- | 2.61-2.73 (2H, m), 3.21-3.27 (2H, m), 3.45 (2H, d), 3.48-3.61 (2H, | | |
| | yl]methylcarbamoylmethylsulfanyl]- | m), 3.74 (2H, d), 3.77-3.86 (1H, m), 7.08-7.15 (1H, m), | | |
| | 3-fluoro-benzoic acid | 7.21-7.25 (1H, m), 7.26-7.33 (1H, m), 7.43-7.51 (1H, m), | | |
| | | 7.63-7.72 (1H, m), 7.76-7.83 (1H, m) | | |
| 17A | 4-[[(2S)-4-[(3,4- | 1.80 (1H, t), 2.02-2.15 (1H, m), 2.55-2.68 (2H, m), 3.25 (2H, | 1.20 | 487/489 |
| | dichlorophenyl)methyl]morpholin-2- | d), 3.42 (2H, s), 3.48-3.61 (2H, m), 3.73-3.75 (2H, m), | | |
| | yl]methylcarbamoylmethylsulfanyl]- | 3.78-3.85 (1H, m), 7.24 (1H, d), 7.45 (1H, d), 7.47-7.52 (2H, m), | | |
| | 3-fluoro-benzoic acid | 7.66-7.72 (1H, m), 7.78-7.83 (1H, m) | | |
| 18A | 2-amino-6-[[(2S)-4-[(3,4- | 1.86 (1H, t), 1.96-2.07 (1H, m), 2.54-2.72 (2H, m), | 0.86 | 451 |
| | difluorophenyl)methyl]morpholin-2- | 3.28-3.35 (2H, m), 3.40 (2H, s), 3.45 (2H, s), 3.57-3.66 (2H, m), | | |
| | yl]methylcarbamoylmethylsulfanyl]pyridine- | 3.84 (1H, d), 6.53 (1H, d), 7.15-7.20 (1H, m), 7.26-7.35 (2H, m), | | |
| | 3-carboxylic acid | 8.04 (1H, d) | | |
| 19A | 2-amino-6-[[(2S)-4-[(4-chloro-3- | 1.77-2.06 (3H, m), 2.30-2.35 (3H, m), 2.54-2.70 (2H, m), | 1.21 | 483/485 |
| | methyl-phenyl)methyl]morpholin-2- | 3.36-3.42 (1H, m), 3.46-3.61 (2H, m), 3.70-3.79 (2H, m), | | |
| | yl]methylcarbamoylmethylsulfanyl]- | 3.88 (1H, dd), 7.07 (1H, d), 7.18 (1H, s), 7.26 (1H, dd), | | |
| | 5-fluoro-pyridine-3-carboxylic acid | 7.74 (1H, dd) | | |
| 20A | 2-amino-6-[[(2S)-4-[(4- | 1.68-1.80 (1H, m), 1.83-1.95 (1H, m), 2.45-2.52 (1H, m), | 1.09 | 469/471 |
| | chlorophenyl)methyl]morpholin-2- | 2.55-2.63 (1H, m), 3.18-3.28 (2H, m), 3.32-3.37 (4H, m), | | |
| | yl]methylcarbamoylmethylsulfanyl]- | 3.45-3.58 (2H, m), 3.67-3.77 (1H, m), 7.26 (4H, dd), | | |
| | 5-fluoro-pyridine-3-carboxylic acid | 7.76 (1H, d) | | |
| 21A | 4-[[(2S)-4-[(4-fluoro-2-methyl- | 1.88 (1H, t), 2.09-2.18 (1H, m), 2.33 (3H, s), 2.58-2.63 (1H, | 0.81 | 434 |
| | phenyl)methyl]morpholin-2- | m), 2.66-2.71 (1H, m), 3.24-3.27 (2H, m), 3.44 (2H, s), | | |
| | yl]methylcarbamoylmethylsulfanyl]pyridine- | 3.46-3.56 (2H, m), 3.76-3.81 (1H, m), 3.84 (2H, d), 6.78-6.85 (1H, | | |
| | 2-carboxylic acid | m), 6.85-6.90 (1H, m), 7.20 (1H, dd), 7.38 (1H, dd), 7.94 (1H, | | |
| | | d), 8.37 (1H, d) | | |
| 22A | 2-amino-5-fluoro-6-[[(2S)-4-[(4- | 1.77 (1H, t), 1.88-1.96 (1H, m), 2.30 (3H, s), 2.45-2.50 (1H, | 1.13 | 467 |
| | fluoro-2-methyl- | m), 2.55-2.60 (1H, m), 3.22-3.29 (2H, m), 3.34 (2H, s), | | |
| | phenyl)methyl]morpholin-2- | 3.45-3.55 (2H, m), 3.70-3.75 (1H, m), 6.76-6.83 (1H, m), | | |
| | yl]methylcarbamoylmethylsulfanyl]pyridine- | 6.83-6.88 (1H, m), 7.12-7.17 (1H, m), 7.76 (1H, d) | | |
| | 3-carboxylic acid | | | |
| 23A | 2-[[(2S)-4-[(4- | 1.64-1.76 (2H, m), 2.39-2.45 (1H, m), 2.54-2.60 (1H, m), | 0.86 | 470 |
| | fluorophenyl)methyl]morpholin-2- | 3.20-3.23 (2H, m), 3.26-3.28 (2H, m), 3.33-3.36 (1H, m), | | |
| | yl]methylcarbamoylmethylsulfanyl]quinoline- | 3.46-3.53 (2H, m), 3.90-4.09 (2H, m), 6.98 (2H, t), | | |
| | 6-carboxylic acid | 7.13-7.18 (2H, m), 7.42 (1H, d), 8.01 (1H, d), 8.21 (1H, d), | | |
| | | 8.25-8.29 (1H, m), 8.55 (1H, s) | | |
| 24A | 2-amino-6-[[(2S)-4-[(4-fluoro-2- | 1.74-1.82 (1H, m), 1.88-1.97 (1H, m), 2.30 (3H, s), | 0.98 | 447 |
| | methyl-phenyl)methyl]morpholin-2- | 2.45-2.60 (2H, m), 3.21-3.29 (2H, m), 3.34 (2H, s), 3.44-3.54 (2H, | | |
| | yl]methylcarbamoylmethylsulfanyl]pyridine- | m), 3.71-3.76 (1H, m), 6.45 (1H, d), 6.77-6.88 (2H, m), | | |
| | 3-carboxylic acid | 7.15 (1H, dd), 7.96 (1H, d) | | |
| | | 2H obscured | | |
| 25A | 6-[[(2S)-4-[(4-fluoro-2-methyl- | 1.78-1.87 (1H, m), 1.99-2.04 (1H, m), 2.32 (3H, s), | 0.82 | 432 |
| | phenyl)methyl]morpholin-2- | 2.52-2.58 (1H, m), 2.60-2.66 (1H, m), 3.22-3.28 (2H, m), 3.38 (2H, | | |
| | yl]methylcarbamoylmethylsulfanyl]pyridine- | s), 3.43-3.53 (2H, m), 3.71-3.78 (1H, m), 3.82 (1H, d), | | |
| | 3-carboxylic acid | 3.92 (1H, d), 6.78-6.90 (2H, m), 7.14-7.19 (1H, m), 7.34 (1H, dd), | | |
| | | 8.12 (1H, dd), 8.98 (1H, dd) | | |
| 26A | 2-amino-6-[[(2S)-4-[(4- | 1.78 (1H, t), 1.88-1.96 (1H, m), 2.49-2.55 (1H, m), | 0.57 | 435 |
| | fluorophenyl)methyl]morpholin-2- | 2.58-2.64 (1H, m), 3.21-3.34 (2H, m), 3.37 (2H, q), 3.46-3.58 (2H, m), | | |
| | yl]methylcarbamoylmethylsulfanyl]pyridine- | 3.68 (1H, d), 3.71-3.77 (1H, m), 3.84 (1H, d), 6.50 (1H, d), | | |
| | 3-carboxylic acid | 7.01 (2H, t), 7.27 (2H, dd), 7.96 (1H, d) | | |
| 27A | 6-[[(2S)-4-[(4- | 1.75 (1H, t), 1.86-1.94 (1H, m), 2.49-2.64 (2H, m), | 0.57 | 420 |
| | fluorophenyl)methyl]morpholin-2- | 3.21-3.27 (2H, m), 3.37 (1H, s), 3.45-3.54 (2H, m), 3.65 (2H, s), | | |
| | yl]methylcarbamoylmethylsulfanyl]pyridine- | 3.73-3.78 (2H, m), 6.98-7.04 (2H, m), 7.26-7.30 (3H, m), 8.10 (1H, | | |
| | 3-carboxylic acid | dd), 8.96-8.98 (1H, m) | | |
| 28A | 6-[[(2S)-4-[(3,4- | 1.75 (1H, t), 1.87-1.95 (1H, m), 2.48-2.61 (2H, m), | 0.75 | 436 (ES−) |
| | difluorophenyl)methyl]morpholin-2- | 3.23-3.27 (2H, m), 3.36 (2H, s), 3.45-3.55 (2H, m), 3.65 (2H, s), | | |
| | yl]methylcarbamoylmethylsulfanyl]pyridine- | 3.73-3.78 (1H, m), 7.03-7.09 (1H, m), 7.13-7.19 (1H, m), | | |
| | 3-carboxylic acid | 7.19-7.24 (1H, m), 7.28 (1H, dd), 8.10 (1H, dd), 8.96-8.98 (1H, m) | | |
| 29A | 2-Amino-6-({[4-(3,4-dichloro- | 1.76 (1H, t), 1.91-1.95 (1H, m), 2.52 (2H, dd), 3.23 (1H, dd), | 1.27 | 485/487 |
| | benzyl)-morpholin-2-ylmethyl]- | 3.35 (2H, s), 3.49-3.58 (3H, m), 3.75 (1H, dt), 6.44 (1H, d), | | |
| | carbamoyl}-methylsulfanyl)-5-fluoro- | 7.20 (1H, dd), 7.41-7.46 (2H, m), 7.96 (1H, d) 2 protons | | |
| | nicotinic acid | obscured | | |
| 32A | 4-[[(2S)-4-[(3,4- | 1.69-1.79 (1H, m), 1.99-2.08 (1H, m), 2.53-2.63 (2H, m), | 0.95 | 455 |
| | difluorophenyl)methyl]morpholin-2- | 3.22 (2H, d), 3.39 (2H, s), 3.46-3.57 (2H, m), 3.65 (2H, s), | | |
| | yl]methylcarbamoylmethylsulfanyl]- | 3.76-3.81 (1H, m), 7.06-7.12 (1H, m), 7.13-7.19 (1H, m), | | |
| | 3-fluoro-benzoic acid | 7.20-7.26 (1H, m), 7.39 (1H, t), 7.64 (1H, dd), 7.73 (1H, dd) | | |
| 33A | 4-[[(2S)-4-[(4- | 1.71-1.79 (1H, m), 1.99-2.07 (1H, m), 2.54-2.65 (2H, m), | 0.78 | 437 |
| | fluorophenyl)methyl]morpholin-2- | 3.19-3.23 (2H, m), 3.40 (1H, s), 3.46-3.57 (2H, m), 3.65 (3H, | | |
| | yl]methylcarbamoylmethylsulfanyl]- | s), 3.76-3.81 (1H, m), 6.99-7.04 (2H, m), 7.28-7.32 (2H, m), | | |
| | 3-fluoro-benzoic acid | 7.39 (1H, t), 7.63 (1H, dd), 7.73 (1H, dd) | | |

### EXAMPLE 34

### Human Eosinophil Chemotaxis

Human eosinophils are isolated from EDTA anticoagulated peripheral blood as previously described (Hansel et al., J. Immunol. Methods, 1991, 145, 105-110). The cells are resuspended at 10×10⁶ mL⁻¹ in RPMI containing 200 IU/mL penicillin, 200 μg/mL streptomycin sulfate and supplemented with 10% HIFCS, at room temperature.

Eosinophils (700 μl) are pre-incubated for 15 mins at 37° C. with 7 μl of either vehicle or compound (100× required final concentration in 10% DMSO). A chemotaxis plate (ChemoTx, 3 μm pore, Neuroprobe) can be loaded by adding 28 μl of a concentration of eotaxin 0.1 to 100 nM (a selective CCR3 agonist over this concentration range) containing a concentration of a compound according to the Examples or solvent to the lower wells of the chemotaxis plate. The filter is then placed over the wells and 25 μl of eosinophil suspension is added to the top of the filter. The plate is incubated for 1 hr at 37° C. in a humidified incubator with a 95% air/5% CO₂ atmosphere to allow chemotaxis.

The medium, containing cells that had not migrated, is carefully aspirated from above the filter and discarded. The filter is then washed once with phosphate buffered saline (PBS) containing 5 mM EDTA to remove any adherent cells. Cells that have migrated through the filter are pelleted by centrifugation (300×g for 5 mins at room temperature) and the filter removed and the supernatant transferred to each well of a 96-well plate (Costar). The pelleted cells are lysed by the addition of 28 μl of PBS containing 0.5% Triton ×100 followed by two cycles of freeze/thawing. The cell lysate is then added to the supernatant. The number of eosinophils migrating can be quantified according to the method of Strath et al., J. Immunol. Methods, 1985, 83, 209 by measuring eosinophil peroxidase activity in the supernatant.

### EXAMPLE 35

### Eotaxin-2-Induced Shape Change in Eosinophils in Human Blood In Vitro

See for example, Differential regulation of eosinophil chemokine signaling via CCR3 and non-CCR3 pathways. Sabroe I, Hartnell A, Jopling L A, Bel S, Ponath P D, Pease J E, Collins P D, Williams T J. J Immunol. 1999 Mar. 1; 162(5):2946-55.

Human blood, collected by venous puncture into 9 mL lithium-heparin tubes, was incubated with the CCR3 agonist eotaxin-2 in the presence of vehicle (0.1% (v/v) DMSO) or test compound for 4 min at 37° C. in a deep, 96-square-well plate. The blood was fixed with Optilyse B (100 μL) at room temperature for 10 min and then the red blood cells were lysed with distilled water (1 mL) for 60 min at room temperature.

The plate was centrifuged at room temperature for 5 min at 300 g. The pellet was re-suspended in assay buffer (PBS without CaCl₂ and MgCl₂, containing HEPES (10 mM), Glucose (10 mM) and 0.1% (w/v) BSA, pH 7.4) and the samples were analysed using flow cytometry (FC500, Beckman Coulter). The high autofluorescence of eosinophils allowed them to be identified as a discrete population from the other blood cell types. Eosinophil shape was monitored as the refractive index of the eosinophil population as determined using the forward scatter signal in flow cytometry.

Eotaxin-2 induced a concentration-dependent change in the forward scatter of eosinophils and these data were used to construct a concentration effect curve (E/[A] curve). The rightward displacement of the eotaxin-2 E/[A] curve in the presence of a CCR3 antagonist was used to estimate a pA₂ value in blood using the following equation:

Single pA₂=−log₁₀([B]/(r−1))

where r is the ratio of the concentrations required for half maximal effects of eotaxin-2 in the absence and presence of antagonist ([A]₅₀ for eotaxin-2 in the presence of antagonist divided by [A]₅₀ for control eotaxin-2 curve) and [B] is the molar concentration of antagonist.

### EXAMPLE 36

### Determination of Compound Affinity at Human Recombinant CCR3 Receptors Assessed by Competition of [³H]-4-(2,4-dichloro-3-methylphenoxy)-1′-[4-(methylsulfonyl)benzoyl]-1,4′-bipiperidine for CHO-K1 Cell Membranes In Vitro

Membranes, prepared from CHO-K1 cells stably expressing recombinant human CCR3, suspended in assay buffer (50 mM Tris-Base, pH 7.4; containing sodium chloride (100 mM) and magnesium chloride (2 mM)) were incubated in the presence of 2 nM [³H]-4-(2,4-dichloro-3-methylphenoxy)-1′-[4-(methylsulfonyl)benzoyl]-1,4′-bipiperidine, along with vehicle (1% (v/v) DMSO), 4-(4-chloro-3-methylphenoxy)-1′-[2-(methylsulfonyl)benzoyl]-1,4′-bipiperidine (to define non-specific binding) or test compound for 2 h at 37° C. in round bottomed 96-well plates. The plates were then filtered onto GF/B filter plates, pre-soaked for 1 hour in plate-coating solution (0.3% (w/v) polyethylenimine, 0.2% (w/v) BSA in de-ionised water), using a 96-well plate Tomtec cell harvester. Four washes (250 μL) with wash buffer (50 mM Tris-Base, pH 7.4 containing sodium chloride (500 mM) and magnesium chloride (2 mM)) were performed at 4° C. to remove unbound radioactivity. Plates were dried and MicroScint-O (50 μL) was added to each well. The plates were sealed (TopSeal A) and filter-bound radioactivity was measured with a scintillation counter (TopCount, Packard BioScience) using a 1 minute counting protocol.

Specific binding was determined from values of the control wells minus the values for the NSB wells for each assay plate. pIC₅₀ values were calculated using a four parameter logistic fit (where pIC₅₀ is defined as the negative logarithm of the concentration of compound required for 50% reduction in specific [³H]-4-(2,4-dichloro-3-methylphenoxy)-1′-[4-(methylsulfonyl)benzoyl]-1,4′-bipiperidine binding). Data were presented as mean pKi values (calculated by applying a Cheng-Prussof correction to pIC₅₀ values) from a minimum of 2 separate experiments.

| Example | CCR3 pKi |
|---|---|
| 2 | 9.4 |
| 6 | 9.3 |
| 8A | 9.7 |
| 9A | 9.6 |
| 10A | 9.7 |
| 16A | 9.6 |
| 29A | 9.4 |
| 31A | 9.0 |

1. A compound of formula (I): 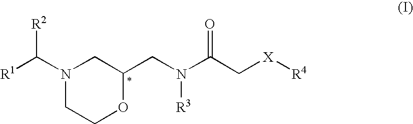
wherein:
R¹ is phenyl optionally substituted by halogen, cyano, C₁₋₄ alkyl or C₁₋₄ alkoxy;
R² and R³ are, independently, hydrogen or C₁₋₆ alkyl;
X is O, S, S(O) or S(O)₂;
R⁴ is phenyl, naphthyl or heteroaryl substituted with CO₂R and optionally further substituted with halogen, hydroxy, nitro, S(O)_{q}(C₁₋₆ alkyl), S(O)₂NH₂, S(O)₂NH(C₁₋₆ alkyl), S(O)₂N(C₁₋₆ alkyl)₂, NH₂, NH(C₁₋₆ alkyl), N(C₁₋₆ alkyl)₂, cyano, C₁₋₆ alkyl, C₁₋₆ alkoxy, C(O)NH₂, C(O)NH(C₁₋₆ alkyl), C(O)N(C₁₋₆ alkyl)₂, CO₂H, CO₂(C₁₋₆ alkyl), NHC(O)(C₁₋₆ alkyl), NHS(O)₂(C₁₋₆ alkyl), C(O)(C₁₋₆ alkyl), CF₃ or OCF₃;
R is hydrogen, C₁₋₆ alkyl or phenyl(C₁₋₄ alkyl); wherein the phenyl is optionally substituted with halogen, hydroxy, nitro, S(O)_{q}(C₁₋₄ alkyl), S(O)₂NH₂, S(O)₂NH(C₁₋₄ alkyl), S(O)₂N(C₁₋₄ alkyl)₂, cyano, C₁₋₄ alkyl, C₁₋₄ alkoxy, C(O)NH₂, C(O)NH(C₁₋₄ alkyl), C(O)N(C₁₋₄ alkyl)₂, CO₂H, CO₂(C₁₋₄ alkyl), NHC(O)(C₁₋₄ alkyl), NHS(O)₂(C₁₋₄ alkyl), C(O)(C₁₋₄ alkyl), CF₃ or OCF₃; or CO₂R is (CO₂⁻)ₚR^{p+} wherein R^{p+} is a univalent cation (for example an alkali metal cation) or two carboxylates may coordinate to a divalent cation (for example an alkaline earth metal cation);
n and q are, independently, 0, 1 or 2;
p is 1 or 2;
or a N-oxide thereof; or a pharmaceutically acceptable salt thereof.

2. A compound of formula (I) as claimed in claim 1 wherein R¹ is phenyl substituted by one, two or three substituents independently selected from: fluorine, chlorine, cyano and methyl.

3. A compound of formula (I) as claimed in claim 1 wherein R² is hydrogen.

4. A compound of formula (I) as claimed in claim 1 wherein R³ is hydrogen.

5. A compound of formula (I) as claimed in claim 1 wherein X is S.

6. A compound of formula (I) as claimed in claim 1 wherein R⁴ is phenyl or pyridinyl substituted with CO₂R and optionally further substituted with halogen, S(O)_{q}(C₁₋₆ alkyl), S(O)₂NH₂, NH₂, NH(C₁₋₆ alkyl), N(C₁₋₆ alkyl)₂, C₁₋₆ alkyl, C₁₋₆ alkoxy, C(O)NH₂, C(O)(C₁₋₆ alkyl), CF₃ or OCF₃; and q is 0, 1 or 2.

7. A compound of formula (I) as claimed in claim 1 wherein R is hydrogen.

8. A compound of formula (I) as claimed in claim 1 where R³ is hydrogen, and the compound of formula (I) has the S stereochemistry at the carbon marked*.

9. A process for preparing a compound of formula (I) as claimed in claim 1, the process comprising:
a. reacting a compound of formula (II): 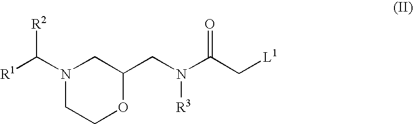
wherein L¹ is a leaving group, with a compound R⁴XH in the presence of a suitable buffer in a suitable solvent at a suitable temperature;
b. reacting a compound of formula (III): 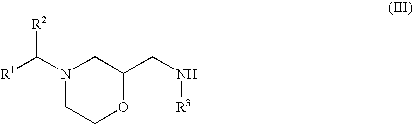
with a compound of formula (IV): 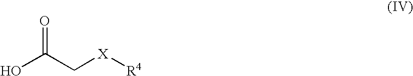
in the presence of a suitable coupling agent, in the presence of a suitable base, in a suitable solvent at a temperature in the range −10 to 30° C.; or,
c. when CO₂R is an ester, reacting a compound of formula (V): 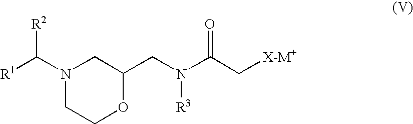
wherein M⁺ is an alkali metal cation, with L²-R⁴, wherein L² is a leaving group, in a suitable solvent at ambient temperature.

10. A pharmaceutical composition which comprises a compound of the formula (I), or a pharmaceutically acceptable salt thereof as claimed in claim 1, and a pharmaceutically acceptable adjuvant, diluent or carrier.

11. (canceled)

12. (canceled)

13. A method of treating a chemokine mediated disease state in a mammal suffering from, or at risk of, said disease, which comprises administering to a mammal in need of such treatment a therapeutically effective amount of a compound of formula (I), or a pharmaceutically acceptable salt thereof as claimed in claim 1.
